(19) United States
(12) Patent Application Publication
(10) Pub. No.: US 2026/0002153 A1
(43) Pub. Date: Jan. 1, 2026

(54) COMPOSITIONS AND METHOD FOR THE TREATMENT OF X-LINKED DYSTONIA PARKINSONISM

(71) Applicant: Jennifer Erwin, Baltimore, MD (US)

(72) Inventors: Jennifer Erwin, Baltimore, CA (US); Apua Paquola, Baltimore, MD (US); Arthur Feltrin, Baltimore, MD (US); Laura D'ignazio, Baltimore, MD (US); Tomoyo Sawada, Baltimore, MD (US)

(73) Assignee: Jennifer Erwin, Baltimore, MD (US)

(21) Appl. No.: 18/856,625

(22) PCT Filed: Apr. 14, 2023
(86) PCT No.: PCT/US23/18661

§ 371 Date: Oct. 13, 2024

Related U.S. Application Data
(60) Provisional application No. 63/330,818, filed on Apr. 14, 2022.

(51) Int. Cl. C12N 15/113; A61P 25/00

(52) U.S. Cl. CPC C12N 15/113; A61P 25/00; C12N 2310/11; C12N 2310/14; C12N 2310/3233

(57) ABSTRACT

Disclosed herein are compositions and methods for treating disease in a subject in need thereof, in some cases administering inhibitory nucleic acids to a patient having X-linked dystonia parkinsonism. Also disclosed herein are compositions and methods relating to the generation of striatal organoids derived from induced pluripotent stem cells.

### CROSS-REFERENCE TO RELATED APPLICATIONS

This application claims the benefit of U.S. Provisional Patent Application No. 63/330,818, filed Apr. 14, 2022. The contents of this application are incorporated herein by reference in their entirety.

### FIELD OF THE INVENTION

Disclosed herein are compositions and methods relating to the study and treatment of X-linked dystonia parkinsonism (XDP). Also disclosed herein are methods of treating XDP.

### SEQUENCE LISTING

This application contains a Sequence Listing that has been submitted electronically as an ASCII text file named 20230525-53760-0002WO1_SL-24309083.The ASCII text file, created on May 25, 2023, is 306 kilobytes in size. The material in the ASCII text file is hereby incorporated by reference in its entirety.

### BACKGROUND

X-linked Dystonia-Parkinsonism (XDP) is an inherited adult-onset neurodegenerative movement disorder of the caudate nucleus caused by a disease-specific SINE/VNTR/Alu (SVA) retrotransposon insertion variant within intron 32 of the TAF1 gene at the Xq13.1 locus. TAF1 encodes the largest component of the TFIID complex, which initiates mRNA transcription by RNA polymerase II. Histopathological analyses indicates a progressive differential degeneration in the adult neostriatum of XDP patients.

SVA elements each include a variable number of hexanucleotide repeats comprising the sequence (CCCTCT)ₙ, and the length of the hexanucleotide repeat sequence within the pathogenic intronic SVA retrotransposon in TAF1 is inversely correlated with age of disease onset. Further, the pathogenic intronic SVA retrotransposon in TAF1 has been shown to interfere with canonical splicing of TAF1 mRNA by promoting retention of intronic sequence proximal to the SVA insertion, resulting in reduced TAF1 expression. However, the mechanism by which the intronic SVA insertion in TAF1 contributes to XDP pathogenesis and causes the neurological phenotype remains unknown. There is currently no cure for XDP and currently available treatments provide only temporary relief to patients.

Therefore, there exists a need for improved compositions and methods for interrogating the molecular pathogenesis of XDP. There also exists a need for improved methods of treating XDP. The methods disclosed herein meet those needs.

### SUMMARY

The compositions and methods disclosed herein are based, at least in part, on the discovery of a novel SVA-derived RNA transcript, a novel method of producing an iPSC-based organoid model of the disease, and therapeutic compositions targeting the SVA-derived RNA transcript.

In one aspect, the compositions and methods disclosed herein include a pharmaceutical composition including an inhibitory nucleic acid that is complementary to a portion of an RNA transcript derived from a SINE/VNTR/Alu (SVA) element. In some implementations, the RNA transcript comprises a hexanucleotide repeat. In some implementations, the hexanucleotide repeat comprises, or is complementary to, the polynucleotide sequence CCCTCT (SEQ ID NO:1). In some implementations, the RNA transcript comprises a polynucleotide sequence derived from the SVA element and a polynucleotide sequence derived from a mammalian genome. In some implementations, the polynucleotide sequence derived from the mammalian genome maps to a protein-coding gene. In some implementations, the polynucleotide sequence derived from the mammalian genome maps to an intron of the protein-coding gene. In some implementations, the protein-coding gene is TAF1 . In some implementations, the mammalian genome is a human genome. In some implementations, the inhibitory nucleic acid is a double stranded RNA species. In some implementations, the inhibitory nucleic acid is a small interfering RNA (siRNA) species. In some implementations, the inhibitory nucleic acid is a morpholino oligomer. In some implementations, the inhibitory nucleic acid is an antisense oligonucleotide. In some implementations, the antisense oligonucleotide is complementary to at least a portion of the 5′-untranslated region (5′UTR) of the RNA transcript. In some implementations, the antisense oligonucleotide comprises a sequence selected from the group consisting of SEQ ID NO:2, SEQ ID NO: 3, SEQ ID NO:4, SEQ ID NO:5, SEQ ID NO:6, SEQ ID NO:7, SEQ ID NO:8, SEQ ID NO: 9, SEQ ID NO: 10, SEQ ID NO: 11, SEQ ID NO:25, and SEQ ID NO:26. In some implementations, the RNA transcript comprises a sequence selected from the group consisting of SEQ ID NO: 12, SEQ ID NO:13, SEQ ID NO: 14, SEQ ID NO: 15, SEQ ID NO: 16, SEQ ID NO:17, SEQ ID NO: 18, SEQ ID NO: 19, SEQ ID NO:20, SEQ ID NO: 21, SEQ ID NO:22, SEQ ID NO:23, and SEQ ID NO:24. In some implementations, the antisense oligonucleotide blocks ribosomes from binding to the RNA transcript. In some implementations, the antisense oligonucleotide interferes with the assembly of the initiation complex at the AUG start codon of the RNA transcript. In some implementations, the antisense oligonucleotide inhibits translation of the RNA transcript. In some implementations, the antisense oligonucleotide comprises one or more phosphorothioate substitutions. In some implementations, the antisense oligonucleotide comprises one or more 2′-O-methyl modifications. In some implementations, the antisense oligonucleotide comprises one or more 2′-O-methoxyethyl modifications. In some implementations, the antisense oligonucleotide comprises one or more 2′-fluoro modifications. In some implementations, the antisense oligonucleotide comprises a sequence of deoxynucleotide monomers flanked by a first sequence of 2′-O modified ribonucleotides at the 5′ end of the antisense oligonucleotide and a second sequence of 2′-O modified ribonucleotides at the 3′ end of the antisense oligonucleotide. In some implementations, the antisense oligonucleotide is conjugated to a peptide species. In some implementations, the antisense oligonucleotide is conjugated to an antibody species. In some implementations, the antisense oligonucleotide is conjugated to an aptamer species. In some implementations, the antisense oligonucleotide forms a duplex with the RNA transcript. In some implementations, the antisense oligonucleotide induces RNAse H-mediated degradation of the RNA transcript. In some implementations, the antisense oligonucleotide is enclosed within a liposome. In some implementations, a plurality of copies of the antisense oligonucleotide are attached to a gold core by metal-thiol linkages to form a spherical nucleic acid nanoparticle. In some implementations, the antisense oligonucleotide is incorporated into the design of a tetrahedron nanostructure to form a self-assembled DNA cage.

In another aspect, the compositions and methods disclosed herein include treating a subject who has a disease caused by the presence of an SVA mobile element in the subject's genome, the method comprising administering to the subject a therapeutically effective amount of an inhibitory nucleic acid that is complementary to a portion of an RNA transcript derived from the SVA element. In some implementations, the RNA transcript comprises a hexanucleotide repeat. In some implementations, the hexanucleotide repeat comprises, or is complementary to, the polynucleotide sequence CCCTCT (SEQ ID NO:1). In some implementations, the RNA transcript comprises a polynucleotide sequence derived from the SVA element and a polynucleotide sequence derived from the subject's genome. In some implementations, the polynucleotide sequence derived from the subject's genome maps to a protein-coding gene. In some implementations, the polynucleotide sequence derived from the subject's genome maps to an intron of the protein-coding gene. In some implementations, the disease caused by the presence of an SVA mobile element in the subject's genome is X-linked Dystonia Parkinsonism. In some implementations, the protein-coding gene is TAF1 . In some implementations, the inhibitory nucleic acid reduces the abundance of the RNA transcript derived from the SVA element. In some implementations, the RNA transcript comprises a sequence selected from the group consisting of SEQ ID NO: 12, SEQ ID NO:13, SEQ ID NO: 14, SEQ ID NO:15, SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, and SEQ ID NO: 24. In some implementations, the inhibitory nucleic acid is an antisense oligonucleotide. In some implementations, the antisense oligonucleotide comprises a sequence selected from the group consisting of SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO:25, and SEQ ID NO:26. In some implementations, the antisense oligonucleotide is enclosed within a liposome. In some implementations, a plurality of copies of the antisense oligonucleotide are attached to a gold core by metal-thiol linkages to form a spherical nucleic acid nanoparticle. In some implementations, the antisense oligonucleotide is incorporated into the design of a tetrahedron nanostructure to form a self-assembled DNA cage. In some implementations, the organoid comprises polarized neuroepithelium-like structures. In some implementations, the organoid is a striatal organoid. In some implementations, the organoid is derived from one or more induced pluripotent stem cells (iPSCs). In some implementations, the one or more iPSCs are derived from a human subject. In some implementations, one or more cells of the organoid express SOX2 and/or NESTIN. In some implementations, one or more cells of the organoid express one or more of DARPP-32, MAP2, and CTIP2. In some implementations, one or more cells of the organoid express CALBINDIN and/or CALRETININ. In some implementations, the human subject is a male having a disorder caused by the presence of an SVA mobile element in the subject's genome. In some implementations, the human subject is a female who is an asymptomatic carrier for a disorder caused by the presence of an SVA mobile element in the subject's genome. In some implementations, the disorder is X-linked Dystonia Parkinsonism (XDP).

In another aspect, the compositions and methods disclosed herein are used in a drug discovery screen, research of etiology of XDP, research of gene expression in XDP, and research of treatments of XDP.

In another aspect, the compositions and methods disclosed herein include a composition comprising an organoid, wherein the organoid comprises polarized neuroepithelium-like structures.

In another aspect, the compositions and methods disclosed herein include inducing formation of a striatal organoid, the method comprising: (i) collecting cells from a subject; (ii) contacting the cells with a DKK-1 and Purmorphamine pathway activator for a period of time sufficient for the cells to differentiate into ventral telencephalic progenitors, neurons and glia; (iii) contacting the cells with a SB 431542 and LDN-193189 for a period of time sufficient for the cells to form neuroepithelium; and (iv) growing the cells in BDNF, GDNF, IGF-1, dibutyryl-CAMP for a period of time sufficient to promote ventral telencephalic progenitors, neurons and glia.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that “the ASO reduces abundance of an RNA molecule transcribed from the TAF1 gene in a cell following administration of the ASO by at least about 60%,” it is implied that the abundance of the RNA molecule is reduced by a range of 50% to 70%.

The term “nucleic acids” or “nucleotides” is intended to encompass plural nucleic acids. In some embodiments, the term “nucleic acids” or “nucleotides” refers to a target sequence, e.g., pre-mRNAs, mRNAs, RNAs, or DNAs in vivo or in vitro. When the term refers to the nucleic acids or nucleotides in a target sequence, the nucleic acids or nucleotides can be naturally occurring sequences within a cell. In other embodiments, “nucleic acids” or “nucleotides” refer to a sequence in the ASOs of the disclosure. When the term refers to a sequence in the ASOs, the nucleic acids or nucleotides are not naturally occurring, i.e., chemically synthesized, enzymatically produced, recombinantly produced, or any combination thereof. In one embodiment, the nucleic acids or nucleotides in the ASOs are produced synthetically or recombinantly, but are not a naturally occurring sequence or a fragment thereof. In another embodiment, the nucleic acids or nucleotides in the ASOs are not naturally occurring because they contain at least one nucleotide analog that is not naturally occurring in nature. The term “nucleic acid” or “nucleoside” refers to a single nucleic acid segment, e.g., a DNA, an RNA, or an analog thereof, present in a polynucleotide. “Nucleic acid” or “nucleoside” includes naturally occurring nucleic acids or non-naturally occurring nucleic acids. In some embodiments, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide (unit) can also be referred to as a monomer or nucleic acid unit. In certain embodiments, the term “nucleotide analogs” refers to nucleotides having modified sugar moieties. Nonlimiting examples of the nucleotides having modified sugar moieties (e.g., LNA) are disclosed elsewhere herein. In other embodiments, the term “nucleotide analogs” refers to nucleotides having modified nucleobase moieties. The nucleotides having modified nucleobase moieties include, but are not limited to, 5-methyl-cytosine, isocytosine, pseudoisocytosine, 5-bromouracil, 5-propynyluracil, 6-aminopurine, 2-aminopurine, inosine, diaminopurine, and 2-chloro-6-aminopurine.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The term “non-coding region” as used herein means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “downstream,” when referring to a nucleotide sequence, means that a nucleic acid or a nucleotide sequence is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence.

In determining the degree of “complementarity” between the ASOs of the disclosure (or regions thereof) and the target region of the nucleic acid transcribed from the TAF1 gene, such as those disclosed herein, the degree of “complementarity” (also, “homology” or “identity”) is expressed as the percentage identity (or percentage homology) between the sequence of the ASO (or region thereof) and the sequence of the target region (or the reverse complement of the target region) that best aligns therewith. The percentage is calculated by counting the number of aligned bases that are identical between the two sequences, dividing by the total number of contiguous monomers in the ASO, and multiplying by 100. In such a comparison, if gaps exist, it is preferable that such gaps are merely mismatches rather than areas where the number of monomers within the gap differs between the ASO of the disclosure and the target region.

The term “complement” as used herein indicates a sequence that is complementary to a reference sequence. It is well known that complementarity is the base principle of DNA replication and transcription as it is a property shared between two

DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′“ATGC”3′ can be written as 3′“TACG”5′ or 5′“GCAT”3′. The terms “reverse complement”, “reverse complementary”, and “reverse complementarity” as used herein are interchangeable with the terms “complement”, “complementary”, and “complementarity.” In some embodiments, the term “complementary” refers to 100% match or complementarity (i.e., fully complementary) to a contiguous nucleic acid sequence within a TAF1 transcript. In some embodiments, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence within a TAF1 transcript.

As used herein, the terms “derived” or “derived from” refers to having originated from, or having been obtained from, a specific source. For example, an SVA-derived RNA transcript is an RNA molecule that originated from an SVA insertion, or whose transcription was initiated due to the presence of the SVA insertion, or that contains SVA polynucleotide sequence. As another example, an “XDP patient-derived striatal organoid” refers to a miniaturized and simplified version of an organ comprising tissue-specific cell-types formed from cells collected from a subject having X-linked dystonia parkinsonism.

As used herein, “plurality” refers to a quantity greater than “1.”

As used herein, “interfere” or “interferes with” refers to a structure or process being blocked, perturbed, or disrupted. For example, an ASO may interfere with the assembly of the translation initiation complex at the AUG start codon of an mRNA transcript by sterically blocking enzymes from accessing the 5′ UTR and AUG start codon of the transcript.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an ASO as disclosed herein is an amount sufficient to carry out a specifically stated purpose. An “effective amount” can be determined empirically and in a routine manner, in relation to the stated purpose.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for a disease or condition disclosed elsewhere herein according to the methods provided herein if the patient shows, e.g., total, partial, or transient alleviation or elimination of symptoms associated with the disease or disorder.

Unless otherwise defined, all technical and scientific terms used herein have the same meaning as commonly understood by one of ordinary skill in the art to which these compositions and methods belong. Although compositions and methods similar or equivalent to those described herein can be used in the practice or testing of the compositions and methods disclosed herein, suitable compositions and methods are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic of the TAF1 locus on the human X chromosome. An SVA insertion is depicted in the antisense orientation in intron 32 of the gene.

FIG. 2A is a schematic of an experimental protocol where cells from an unaffected healthy control individual, cells from a patient having XDP, cells from a patient having XDP wherein the SVA insertion in TAF1 has been excised by CRISPR/Cas9, and cells wherein non-editing CRISPR/Cas9 reagents were used, are assayed for presence/absence of the SVA insertion in the TAF1 gene using long-range PCR primers flanking the expected location of the SVA insertion.

FIG. 2B is a schematic of an experimental protocol where cells from an unaffected healthy control individual, cells from a patient having XDP, cells from a patient having XDP wherein the SVA insertion in TAF1 has been excised by CRISPR/Cas9, and cells wherein non-editing CRISPR/Cas9 reagents were used, are assayed for presence/absence of the SVA insertion in the TAF1 gene using long-range PCR primers flanking the expected location of the SVA insertion.

FIG. 2C is an image of an agarose gel where PCR products of the experiment depicted in the schematic of FIGS. 2A-2B have been separated by electrophoresis. The expected product size for individuals harboring the SVA insertion is approximately 3 kilobases, and the expected product size for individuals not harboring the SVA insertion is approximately 500 base-pairs.

FIG. 3 is a schematic depicting the protocol for generating striatal organoids from patient-derived induced pluripotent stem cells (iPSCs).

FIG. 4A is a series of images produced by fluorescent microscopy of striatal organoids generated from patient-derived iPSCs after 30 days of organotypic culture. Images represent organoids generated from cells from an unaffected healthy control individual, cells from a patient having XDP, and cells from a patient having XDP wherein the SVA insertion in TAF1 has been excised by CRISPR/Cas9. Tissues were stained for the following markers: Ki-67, ZO-1, FOXG1, MEIS2, SOX2, NESTIN, and MAP.

FIG. 4B is a series of images produced by fluorescent microscopy of striatal organoids generated from patient-derived iPSCs after 120 days of organotypic culture. Images represent organoids generated from cells from an unaffected healthy control individual, cells from a patient having XDP, and cells from a patient having XDP wherein the SVA insertion in TAF1 has been excised by CRISPR/Cas9. Tissues were stained for the following markers: CTIP2, GABA, MAP2, CALB2, CALB1, MA, GAD67, MAP2, and DARPP.

FIG. 5 is a plot showing relative expression of an RNA transcript from the TAF1 gene where the presence of an SVA insertion causes partial retention of the proximal segment of intron 32 and multiple aberrant splicing events, termed TAF1-32i. Relative expression of TAF1-32i is shown for iPSCs and striatal organoids generated from an unaffected healthy control individual, cells from a patient having XDP, and cells from a patient having XDP wherein the SVA insertion in TAF1 has been excised by CRISPR/Cas9. Expression of TAF1-32i was quantified at 30 days, 60 days, 90 days, and 120 days of organotypic culture.

FIG. 6A is a schematic of the TAF1 gene, with primer binding sites for the detection of the TAF1-32i RNA transcript indicated. TAF1-5′-F and TAF1-5′-R primers amplify a portion of the 5′ region of the transcript, and TAF1-3′-F and TAF1-3′-R amplify a portion of the 3′ region of the transcript. In a qPCR assay, the TAF1-3′/5′ ratio can be calculated.

FIG. 6B is a plot showing the TAF1-3′/5′ ratio as quantified by qPCR in XDP iPSCs and delSVA-XDP iPSCs (where the SVA insertion has been excised by CRISPR/Cas9).

FIG. 7A is a schematic of the TAF1 locus indicating where oligonucleotide primers were designed to detect SVA-derived RNA transcripts.

FIG. 7B is a plot of relative TAF1-SVA expression for iPSCs and iPSC-derived striatal organoids generated from XDP patients and unaffected healthy controls.

FIG. 8 is a schematic of eleven antisense oligonucleotides designed to target the SVA-derived RNA transcript of the TAF1 gene. The ASOs comprise modified “gapmer” oligonucleotides with a modified phosphorothioate backbone.

FIG. 9 is a schematic of eleven antisense oligonucleotides designed to target the SVA-derived RNA transcript of the TAF1 gene. The ASOs comprise modified oligonucleotides with a modified phosphorothioate backbone.

FIG. 10A is a fluorescent image and plot of quantification of immunohistochemistry for Cleaved Caspase 3, an apoptotic marker, in 120-day XDP (XDP) and isogenic control organoids (ΔSVA-XDP).

FIG. 10B is a fluorescent image and plot of quantification of immunohistochemistry for yH2AX, a DNA damage marker, in 120-day XDP (XDP) and isogenic control organoids (ΔSVA-XDP).

FIG. 10C is a fluorescent image and plot of quantification of immunohistochemistry for 53BP1, a DNA damage marker, in 120-day XDP (XDP) and isogenic control organoids (ΔSVA-XDP).

FIG. 10D is a microscopy image of in situ hybridization of RNA aggregates in 120-day XDP organoids (XDP).

FIG. 10E is a microscopy image of in situ hybridization of RNA aggregates in isogenic control organoids (Del-SVA, 33363.D-3C2).

FIG. 10F is a plot of mean firing rate as measured by multielectrode array (MEA) in 120-day XDP (XDP) and isogenic control organoids (ΔSVA-XDP).

FIG. 10G is a plot of burst intensity as measured by multielectrode array (MEA) in 120-day XDP (XDP) and isogenic control organoids (ΔSVA-XDP).

FIG. 10H is a plot of synchrony index as measured by multielectrode array (MEA) in 120-day XDP (XDP) and isogenic control organoids (ΔSVA-XDP).

FIG. 10I is a plot of number of bursts as measured by multielectrode array (MEA) in 120-day XDP (XDP) and isogenic control organoids (ΔSVA-XDP).

FIG. 11 shows a schematic of the RT-qPCR assay used to measure the abundance of TAF1 SVA-derived transcripts (top), a plot of abundance of the TAF1 SVA-derived transcript in induced pluripotent stem cells (iPSCs) and organoids (lower left), and a plot of abundance of the TAF1 SVA-derived transcript in post-mortem brain tissue collected from six XDP patients (lower right).

FIG. 12 is a plot of TAF1 XDP SVA transcript expression normalized to GAPDH in XDP ventral forebrain organoids after treatment with the ASOs disclosed herein.

FIG. 13A is a plot of coverage of the first base of long-read RNA-Seq reads (PacBio Iso-Seq) in human brain (ENCODE data, T2T-CHM13). FIG. 13B is a plot of 5′ hexamer repeat length of individual SVA loci by type.

### DETAILED DESCRIPTION

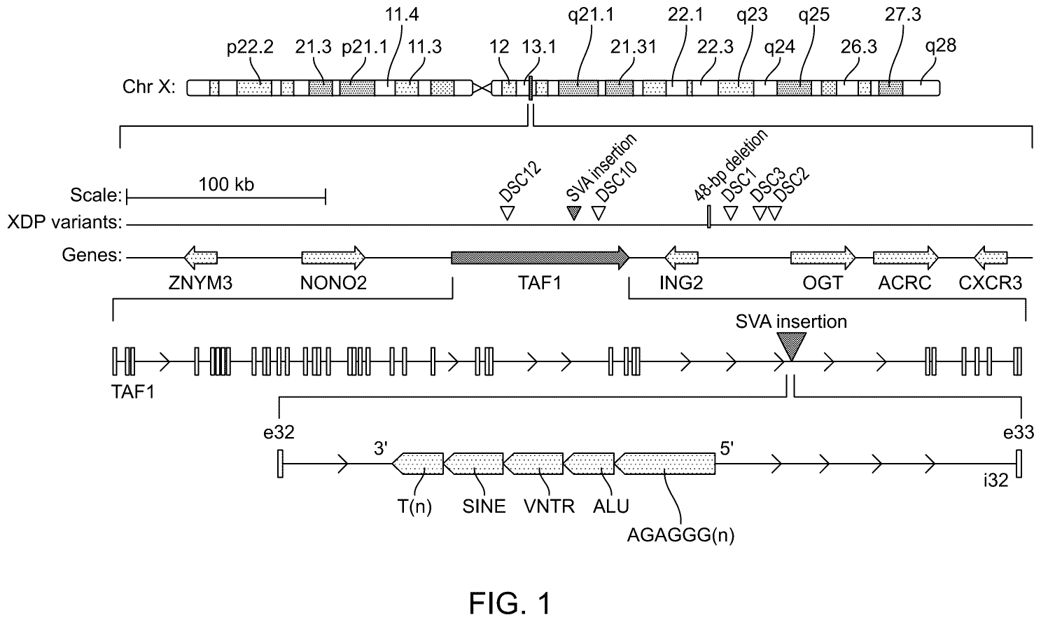

X-linked Dystonia-Parkinsonism (XDP) is an inherited adult-onset neurodegenerative movement disorder of the caudate nucleus affecting predominantly male of Filipino descent. At a molecular level, a disease-specific SINE-VNTR-Alu (SVA) retrotransposon insertion occurs within intron 32 of TAF1 at the Xq13.1 locus (see, e.g., FIG. 1), a core member of the TFIID complex mediating the RNA pol II promoter initiation, and the length of the (CCCTCT)ₙ hexamer repeat within the pathogenic SVA retrotransposon is inversely correlated with age of disease onset. Histopathological analyses indicate a progressive differential degeneration in the adult neostriatum of XDP patients. However, the mechanism by which the intronic SVA insertion in TAF1 contributes to XDP pathogenesis and causes the neurological phenotype remains unknown. There is currently no cure for XDP and currently available treatments provide only temporary relief to patients.

Since about the 1990s, classical human genetic linkage analysis had mapped the disease-causing locus for inherited XDP to a segment of the X chromosome. (Kupke KG, et al. Dystonia-parkinsonism syndrome (XDP) locus: flanking markers in Xq12-q21.1.Am J Hum Genet. 1992 April;50(4):808-15.) More recently, sequencing studies and fine-mapping identified an SVA insertion in intron 32 of the TAF1 gene and decreased expression levels of the TAF1 protein as possible causative mechanism for the XDP phenotype. (Makino S, et al. Reduced neuron-specific expression of the TAF1 gene is associated with X-linked dystonia-parkinsonism. Am J Hum Genet. 2007 March;80(3):393-406.doi: 10.1086/512129.) Finally, genome assembly and transcriptomic studies identified altered splicing and intron retention within the TAF1 gene as possible causative mechanisms for the disease. (Aneichyk T, et al. Dissecting the Causal Mechanism of X-Linked Dystonia-Parkinsonism by Integrating Genome and Transcriptome Assembly. Cell. 2018 Feb. 22; 172(5):897-909.e21.) However, the causal pathway from mRNA splicing to clinical phenotype, and any possible treatments for XDP, remain unknown.

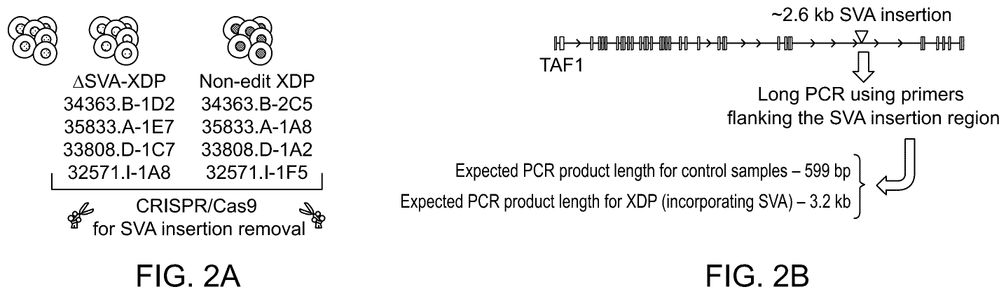

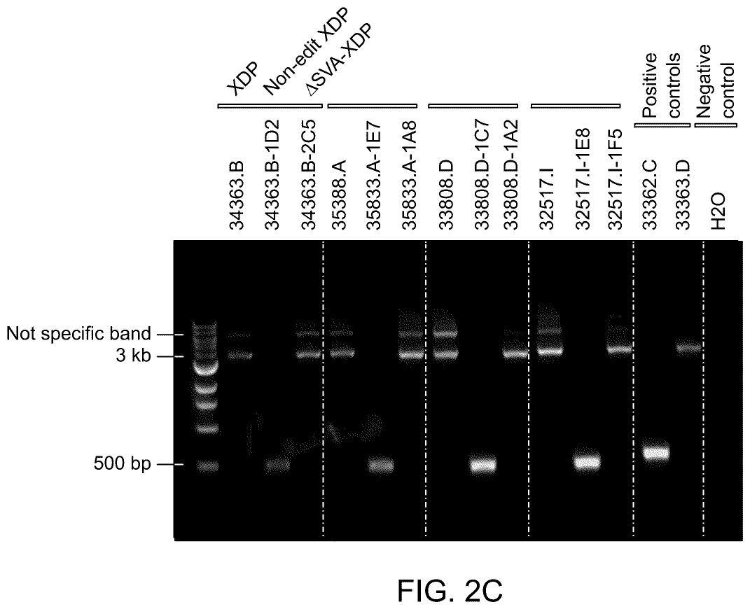

SVA retrotransposons are a class of composite mobile elements that remain active in human genomes. SVA insertions may range in size from 700-4000 basepairs (bp). Starting at the 5′ end, SVA elements include (1) a hexameric CCCTCT repeat, (2) sequence sharing homology to two antisense Alu fragments, (3) a variable number of GC-rich tandem repeats (VNTRs), (4) sequence sharing identity to the env gene and right LTR of an ancient endogenous retrovirus, HERV-K10, followed by (5) a canonical polyadenylation signal (polyA), AATAAA. (Hancks DC, Kazazian HH Jr. SVA retrotransposons: Evolution and genetic instability. Semin Cancer Biol. 2010 August;20(4):234-45.) With respect to human disease, SVA elements may have a functional impact on the genome region in which they reside, at the DNA, RNA, and epigenetic levels. In the case of polymorphic insertions (where any individual human may harbor a presence or absence of the insertion, e.g., in the case of XDP an inherited SVA insertion in the TAF1 gene, see FIGS. 2A-2C) these insertions may contribute to human phenotypic variability including disease. SVAs may contain the inherent ability to mediate transcriptional initiation upstream of their genomic location due to an internal enhancer element. SVA insertions within protein-coding genes may also mediate exon-skipping or be associated with loss of mRNA expression due to exon-trapping, exonization of the SVA elements itself, either mechanism triggering nonsense-mediated decay of mRNA transcripts.

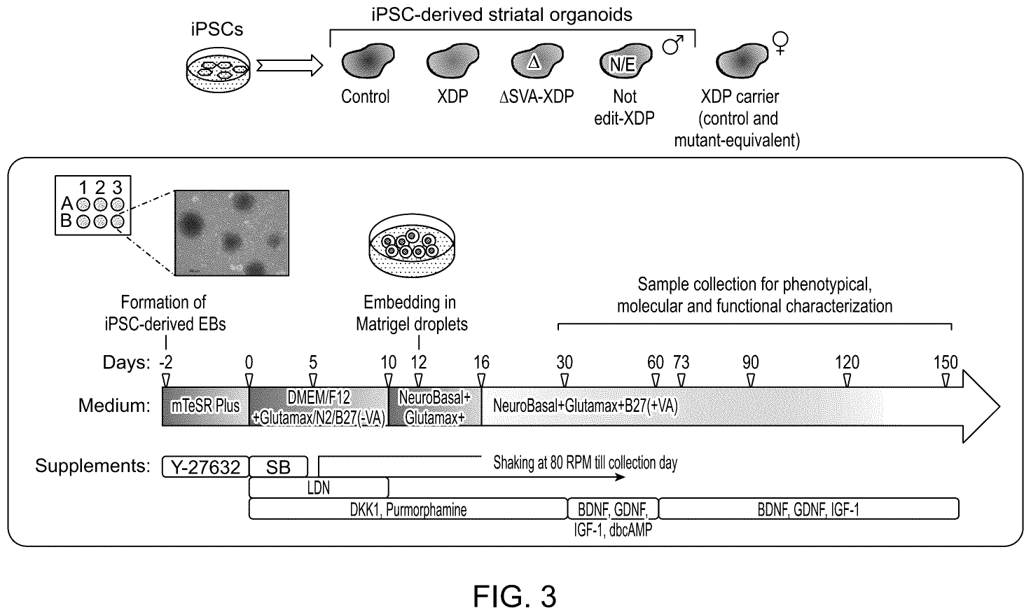

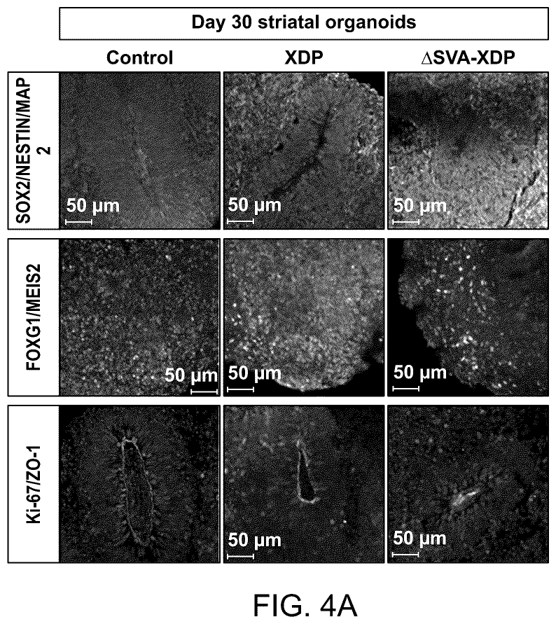

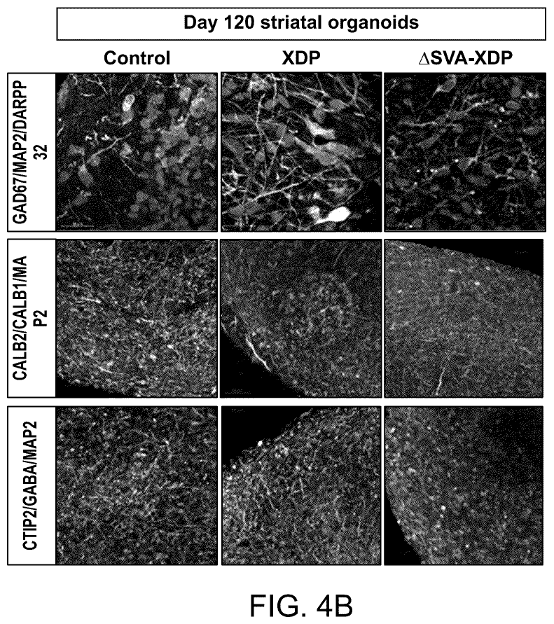

Applicants have developed a novel protocol to differentiate female carrier and male control, XDP-derived, and isogenic SVA-deleted XDP induced pluripotent stem cells (iPSCs) into striatal organoids (see, e.g. FIG. 3). Through extensive phenotypic, molecular, and transcriptomic analysis and single-cell RNA sequencing (see FIGS. 4-6), applicants have found that striatal organoids from XDP patients demonstrate decreased calretinin expression, somatic DNA expansion of the hexamer repeat during organoid maturation, decreased full-length TAF1 expression, and apoptosis. Applicants have identified a novel SVA-derived transcript that may mediate XDP phenotypes in patients harboring the pathogenic intronic SVA insertion in the TAF1 gene. Beyond XDP, the (CCCTCT)n hexamer repeat is embedded in most human genomic SVA retrotransposon insertions, which are present as over 1,000 other copies dispersed throughout the human genome and are the major source of primate specific variable nucleotide repeat polymorphisms in humans.

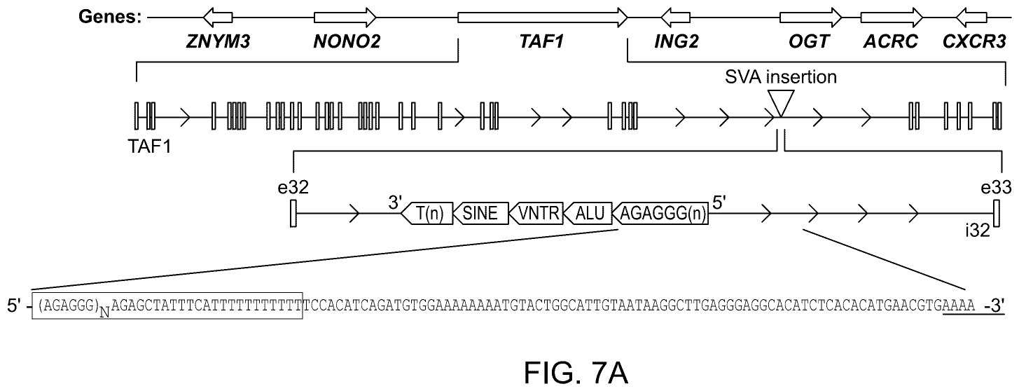

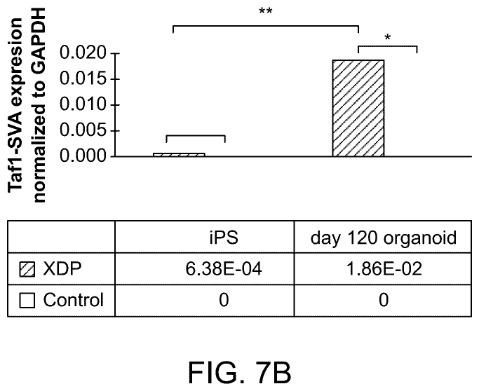

Applicants have also developed inhibitory nucleic acids that form the basis of therapeutic pharmaceutical compositions for the treatment of diseases mediated by SVA insertion and/or RNA transcripts containing variable numbers of hexanucleotide repeats (see FIG. 7). Further, when polymorphic insertions are present within the coding or noncoding regulatory sequences associated with protein-coding genes, the variable nucleotide repeat polymorphisms are known to affect the expression of proximate genes. Therefore, the therapeutic strategies disclosed herein may be applicable to various disease phenotypes caused by SVA retrotransposon insertions or hexanucleotide repeats.

### Pharmaceutical Compositions

Disclosed herein are pharmaceutical compositions directed to inhibitory nucleic acids for use in treating a human subject. In some embodiments, the inhibitory nucleic acids are oligonucleotides. In some embodiments, the inhibitory nucleic acids are modified oligonucleotides. In some embodiments, the inhibitory nucleic acids are antisense oligonucleotides. In some embodiments, the modified oligonucleotides are antisense oligonucleotides that hybridize to an RNA transcript. In some embodiments, the modified oligonucleotides are antisense oligonucleotides that hybridize to an RNA transcript containing a variable number of hexanucleotide repeats. In some embodiments, the modified oligonucleotides are antisense oligonucleotides that hybridize to an RNA transcript that contains an SVA insertion. In some embodiments, the modified oligonucleotides are antisense oligonucleotides that hybridize to an RNA transcript of the TAF1 gene that contains an SVA insertion.

In some embodiments, the pharmaceutical compositions can achieve a therapeutic effect, e.g., reducing or alleviating the symptoms of a disease in a patient. In some embodiments, the disease is a genetic disorder. In some embodiments, the disease is caused by one more inherited mutations of a gene. In some embodiments, the inherited mutation is a retrotransposon insertion mutation. In embodiments, the retrotransposon insertion is an SVA element insertion. In some embodiments, the disease is caused by an RNA transcript comprising a hexanucleotide repeat. In some embodiments the disease is caused by an RNA transcript comprising a hexanucleotide repeat within an SVA element. In some embodiments the disease is caused by an RNA transcript comprising one or repeats of the hexanucleotide sequence CCCTCT (SEQ ID NO:1). In some embodiments, the disease is caused by one or more mutations of the gene TAF1. In some embodiments, the disease is X-linked Dystonia Parkinsonism. In some embodiments, the therapeutic effect includes a reduction or elimination of symptoms including dystonia; resting tremor; bradykinesia; rigidity; postural instability; severe shuffling gait; and dystonia of the jaw, neck, trunk, eyes, limbs, tongue, pharynx, or larynx; among other symptoms of disease.

In some embodiments, the pharmaceutical composition comprises an inhibitory nucleic acid, e.g., an antisense oligonucleotide that is complementary to one or more RNA transcripts that may cause the disease in the patient. In some embodiments, as described in further detail below, the antisense oligonucleotide includes different modifications, e.g., in the sugar backbone to make it more cell permeable and nuclease resistant and physiologically non-toxic at low concentrations.

Inhibitory nucleic acids useful in the present methods and compositions include antisense oligonucleotides, modified bases/locked nucleic acids (LNAs), antagomirs, peptide nucleic acids (PNAs), double stranded RNA species, siRNAs, morpholinos, and other oligomeric compounds or oligonucleotide mimetics which hybridize to at least a portion of the target nucleic acid (i.e., an RNA transcript transcribed from the TAF1 gene) and modulate its abundance, splicing, post-transcriptional processing, or translation; see, e.g., U.S. Pat. Nos. 9,045,749 and 9,476,046. In some embodiments, the inhibitory nucleic acids include antisense RNA, antisense DNA, chimeric antisense oligonucleotides, antisense oligonucleotides comprising modified linkages, or combinations thereof. See, e.g., WO 2010040112.

In some embodiments, the inhibitory nucleic acids are 9 to 50, 9 to 21, 13 to 50, or 13 to 30 nucleotides in length. One having ordinary skill in the art will appreciate that this embodies oligonucleotides having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therein. In some embodiments, the antisense oligonucleotides are 15 nucleotides in length. In some embodiments, the antisense oligonucleotides are 12 or 13 to 30 nucleotides in length. One having ordinary skill in the art will appreciate that this embodies inhibitory nucleic acids having antisense portions of 12, 13, 14, 15, 16, 17, 18, 19, 20, 21, 22, 23, 24, 25, 26, 27, 28, 29 or 30 nucleotides in length, or any range therein.

In some embodiments, the inhibitory nucleic acids are designed to target a specific region of an RNA transcript. For example, a specific functional region can be targeted, e.g., a region proximate to a transcription start site, exon-intron-boundaries, a portion of a hexanucleotide repeat, or a portion of an SVA element.

In some embodiments, the inhibitory nucleic acids are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22:344-359; Nowotny et al., Cell, 121:1005-1016, 2005; Kurreck, European Journal of Biochemistry 270:1628-1644, 2003; Fluiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 372( ) 137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH—N(CH₃)—O—CH₂ (known as a methylene(methylimino) or MMI backbone, CH₂—O—N—(CH₃)—CH₂, CH₂—N—(CH₃)—N (CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃, OCH₃, OCH₃, O(CH₂),CH₃, O(CH₂)ₙNH₂ or O(CH₂)ₙCH₃ where n is from 1 to about 10; Ci to C10 lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, Helv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine and 2,6-diaminopurine. Kornberg, A., DNA Replication, W. H. Freeman & Co., San Francisco, 1980, pp75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, a palmityl moiety, or an octadecylamine or hexylamino-carbonyl-t oxycholesterol moiety.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention.

The inhibitory nucleic acids useful in the present compositions and methods are sufficiently complementary to all or part of a target RNA transcript, i.e., hybridize sufficiently well and with sufficient specificity, to give the desired therapeutic effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a target RNA transcript, then the bases are considered to be complementary to each other at that position. 100% complementarity is not required.

In the context of the present compositions and methods, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. The inhibitory nucleic acids and the target RNA transcript are complementary to each other when a sufficient number of corresponding positions in each molecule are occupied by nucleotides that can hydrogen bond with each other. Thus, “specifically hybridizable” and “complementary” are terms which are used to indicate a sufficient degree of complementarity or precise pairing such that stable and specific binding occurs between the inhibitory nucleic acid and the target RNA transcript. For example, if a base at one position of an inhibitory nucleic acid is capable of hydrogen bonding with a base at the corresponding position of the target RNA transcript, then the bases are considered to be complementary to each other at that position.

Although in some embodiments, 100% complementarity is desirable, it is understood in the art that a complementary nucleic acid sequence need not be 100% complementary to that of its target nucleic acid to be specifically hybridisable. A complementary nucleic acid sequence for purposes of the present compositions and methods is specifically hybridisable when binding of the sequence to the target RNA transcript interferes with the abundance, splicing, post-transcriptional processing, or translation of the RNA transcript, and there is a sufficient degree of complementarity to avoid non-specific binding of the sequence to non-target nucleic acid molecules under conditions in which specific binding is desired, e.g., under physiological conditions.

In general, the inhibitory nucleic acids useful in the compositions and methods described herein have at least 80% sequence complementarity to a target region within the target nucleic acid, e.g., 90%, 91%, 92%, 93%, 94%, 95%, 96%, 97%, 98%, 99% or 100% sequence complementarity to the target region within miR-128 (e.g., a target region comprising the seed sequence). For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Antisense and other compounds of the invention that hybridize to a miR-128 target sequence are identified through routine experimentation. In general the inhibitory nucleic acids must retain specificity for their target, i.e., must not directly bind to, or directly significantly affect expression levels of, transcripts other than the intended target RNA transcript.

In some embodiments, the inhibitory nucleic acids are antisense oligonucleotides. Antisense oligonucleotides are typically designed to inhibit expression of a DNA or RNA target by binding to the target and halting expression at the level of transcription, affecting RNA stability, translation, or splicing. Antisense oligonucleotides of the present compositions and methods are complementary nucleic acid sequences designed to a target RNA transcript to achieve a therapeutic effect in a patient. Thus, oligonucleotides are chosen that are sufficiently complementary to the target, i.e., that hybridize sufficiently well and with sufficient specificity, to give the desired therapeutic effect.

In some embodiments, the ASO is capable of reducing the abundance of an RNA transcribed from the TAF1 gene. In some embodiments, the ASO is capable of reducing the abundance of a TAF1 RNA in a human cell (e.g., HEK293 cell, an iPSC cell). In some embodiments, the TAF1 RNA expression is reduced by at least about 30%, by at least about 35%, by at least about 40%, by at least about 45%, by at least about 50%, by at least about 55%, by at least about 60%, by at least about 65%, by at least about 70%, by at least about 75%, by at least about 80%, by at least about 85%, by at least about 90%, by at least about 95%, or about 100% compared to the TAF1 RNA abundance in a human cell that is not exposed to the ASO. In some embodiments, the ASO is capable of reducing the abundance the TAF1-32i RNA transcript.

In some embodiments, the ASO is capable of reducing the TAF1 transcript (e.g., RNA) expression in a human cell (e.g., HEK293 cell, an iPSC cell). In some embodiments, the TAF1 transcript expression is reduced by at least about 30%, by at least about 35%, by at least about 40%, by at least about 45%, by at least about 50%, by at least about 55%, by at least about 60%, by at least about 65%, by at least about 70%, by at least about 75%, by at least about 80%, by at least about 85%, by at least about 90%, by at least about 95%, or about 100% compared to TAF1 transcript expression in a human cell that is not exposed to the ASO. In some embodiments the ASO is capable of reducing the TAF1-3′/5′ ratio in a human cell. In some embodiments the ASO is capable of increasing the TAF1-3′/5′ ratio in a human cell.

In some embodiments, the pharmaceutical composition is an antisense oligonucleotide comprising the sequence AAATGAAATAGCTCTCCCTC (SEQ ID NO: 2); AAAATGAAATAGCTCTCCCT (SEQ ID NO:3); AATGAAATAGCTCTCCCTCT (SEQ ID NO:4); ATGAAATAGCTCTCCCTCTC (SEQ ID NO: 5); AAAAATGAAATAGCTCTCCC (SEQ ID NO:6); AAAAAATGAAATAGCTCTCC (SEQ ID NO:7); TTTTTTCCACATCTGATGTG (SEQ ID NO: 8); GCCTTATTACAATGCCAGTA (SEQ ID NO:9); CTCAAGCCTTATTACAATGC (SEQ ID NO:10); CCCTCAAGCCTTATTACAAT (SEQ ID NO:11); CCCTCTCCCTCTCCCTCTCC (SEQ ID NO:25); or CCCTCTCCCTCT (SEQ ID NO: 26). In some embodiments, the pharmaceutical composition is an antisense oligonucleotide comprising any of SEQ ID NO:2, SEQ ID NO: 3, SEQ ID NO:4, SEQ ID NO:5, SEQ ID NO:6, SEQ ID NO:7, SEQ ID NO:8, SEQ ID NO: 9, SEQ ID NO: 10, SEQ ID NO: 11, SEQ ID NO: 25, or SEQ ID NO: 26, wherein all or some of the nucleotides have been modified in one or more ways as described above, e.g., with a phosphorothioate backbone, one or more 2′-methoxyethoxy modifications, one or more 2′-methoxy modifications, one or more 2′-propoxy modifications, or one or more 2′-fluoro modifications.

In some embodiments, the pharmaceutical composition is an antisense oligonucleotide comprising the sequence

(SEQ ID NO: 27)
52MOErA/*/i2MOErA/*/i2MOErA/*/i2MOErT/*/i2MOErG/*A*A*A*T*A*G*C*T*C
*T*/i2MOErC/*/i2MOErC/*/i2MOErC/*/i2MOErT/*/32MOErC;
(SEQ ID NO: 28)
52MOErA/*/i2MOErA/*/i2MOErA/*/i2MOErA/*/i2MOErT/*G*A*A*A*T*A*G*C*T
*C*/i2MOErT/*/i2MOErC/*/i2MOErC/*/i2MOErC/*/32MOErT;
(SEQ ID NO: 29)
52MOErA/*/i2MOErA/*/i2MOErT/*/i2MOErG/*/i2MOErA/*A*A*T*A*G*C*T*C*T*
C*/i2MOErC/*/i2MOErC/*/i2MOErT/*/i2MOErC/*/32MOErT;
(SEQ ID NO: 30)
52MOErA/*/i2MOErT/*/i2MOErG/*/i2MOErA/*/i2MOErA/*A*T*A*G*C*T*C*T*C*
C*/i2MOErC/*/i2MOErT/*/i2MOErC/*/i2MOErT/*/32MOErC;
(SEQ ID NO: 31)
52MOErA/*/i2MOErA/*/i2MOErA/*/i2MOErA/*/i2MOErA/*T*G*A*A*A*T*A*G*C
*T*/i2MOErC/*/i2MOErT/*/i2MOErC/*/i2MOErC/*/32MOErC;
(SEQ ID NO: 32)
52MOErA/*/i2MOErA/*/i2MOErA/*/i2MOErA/*/i2MOErA/*A*T*G*A*A*A*T*A*G
*C*/i2MOErT/*/i2MOErC/*/i2MOErT/*/i2MOErC/*/32MOErC;
(SEQ ID NO: 33)
52MOErT/*/i2MOErT/*/i2MOErT/*/i2MOErT/*/i2MOErT/*T*C*C*A*C*A*T*C*T*
G*/i2MOErA/*/i2MOErT/*/i2MOErG/*/i2MOErT/*/32MOErG;
(SEQ ID NO: 34)
52MOErG/*/i2MOErC/*/i2MOErC/*/i2MOErT/*/i2MOErT/*A*T*T*A*C*A*A*T*G*
C*/i2MOErC/*/i2MOErA/*/i2MOErG/*/i2MOErT/*/32MOErA;
(SEQ ID NO: 35)
52MOErC/*/i2MOErT/*/i2MOErC/*/i2MOErA/*/i2MOErA/*G*C*C*T*T*A*T*T*A*
C*/i2MOErA/*/i2MOErA/*/i2MOErT/*/i2MOErG/*/32MOErC;
(SEQ ID NO: 36)
52MOErC/*/i2MOErC/*/i2MOErC/*/i2MOErT/*/i2MOErC/*A*A*G*C*C*T*T*A*T*
T*/i2MOErA/*/i2MOErC/*/i2MOErA/*/i2MOErA/*/32MOErT;
or
(SEQ ID NO: 37)
52MOErC/*/i2MOErC/*/i2MOErC/*/i2MOErT/*/i2MOErC/*T*C*C*C*T*C*T*C*C*
C*/i2MOErT/*/i2MOErC/*/i2MOErT/*/i2MOErC/*/32MOErC,
wherein “52MOEr” indicates a 5′-terminal 2′-O-methoxyethyl modification, “i2MOEr”
indicates an internal 2′-O-methoxyethyl modification, “32MOEr” indicates a 3′-terminal
2′-O-methoxyethyl modification, and an asterisk indicates the presence of a
phosphorothioate internucleoside linkage.

In some embodiments, the pharmaceutical composition is an antisense oligonucleotide comprising the sequence

(SEQ ID NO: 38)
52MOErA/*/i2MOErA/*/i2MOErA/*/i2MOErT/*/i2MOErG/*/i2MOErA/*/i2MOErA/*/
i2MOErA/*/i2MOErT/*/i2MOErA/*/i2MOErG/*/i2MOErC/*/i2MOErT/*/i2MOErC/*/
i2MOErT/*/i2MOErC/*/i2MOErC/*/i2MOErC/*/i2MOErT/*/32MOErC;
(SEQ ID NO: 39)
52MOErA/*/i2MOErA/*/i2MOErA/*/i2MOErA/*/i2MOErT/*/i2MOErG/*/i2MOErA/*/
i2MOErA/*/i2MOErA/*/i2MOErT/*/i2MOErA/*/i2MOErG/*/i2MOErC/*/i2MOErT/*/
i2MOErC/*/i2MOErT/*/i2MOErC/*/i2MOErC/*/i2MOErC/*/32MOErT;
(SEQ ID NO: 40)
52MOErA/*/i2MOErA/*/i2MOErT/*/i2MOErG/*/i2MOErA/*/i2MOErA/*/i2MOErA/*/
i2MOErT/*/i2MOErA/*/i2MOErG/*/i2MOErC/*/i2MOErT/*/i2MOErC/*/i2MOErT/*/
i2MOErC/*/i2MOErC/*/i2MOErC/*/i2MOErT/*/i2MOErC/*/32MOErT;
(SEQ ID NO: 41)
52MOErA/*/i2MOErT/*/i2MOErG/*/i2MOErA/*/i2MOErA/*/i2MOErA/*/i2MOErT/*/
i2MOErA/*/i2MOErG/*/i2MOErC/*/i2MOErT/*/i2MOErC/*/i2MOErT/*/i2MOErC/*/
i2MOErC/*/i2MOErC/*/i2MOErT/*/i2MOErC/*/i2MOErT/*/32MOErC;
(SEQ ID NO: 42)
52MOErA/*/i2MOErA/*/i2MOErA/*/i2MOErA/*/i2MOErA/*/i2MOErT/*/i2MOErG/*/
i2MOErA/*/i2MOErA/*/i2MOErA/*/i2MOErT/*/i2MOErA/*/i2MOErG/*/i2MOErC/*/
i2MOErT/*/i2MOErC/*/i2MOErT/*/i2MOErC/*/i2MOErC/*/32MOErC;
(SEQ ID NO: 43)
52MOErA/*/i2MOErA/*/i2MOErA/*/i2MOErA/*/i2MOErA/*/i2MOErA/*/i2MOErT/*/
i2MOErG/*/i2MOErA/*/i2MOErA/*/i2MOErA/*/i2MOErT/*/i2MOErA/*/i2MOErG/*/
i2MOErC/*/i2MOErT/*/i2MOErC/*/i2MOErT/*/i2MOErC/*/32MOErC;
(SEQ ID NO: 44)
52MOErT/*/i2MOErT/*/i2MOErT/*/i2MOErT/*/i2MOErT/*/i2MOErT/*/i2MOErC/*/
i2MOErC/*/i2MOErA/*/i2MOErC/*/i2MOErA/*/i2MOErT/*/i2MOErC/*/i2MOErT/*/
i2MOErG/*/i2MOErA/*/i2MOErT/*/i2MOErG/*/i2MOErT/*/32MOErG;
(SEQ ID NO: 45)
52MOErG/*/i2MOErC/*/i2MOErC/*/i2MOErT/*/i2MOErT/*/Ai2MOEr/*/i2MOErT/*/
i2MOErT/*/i2MOErA/*/i2MOErC/*/i2MOErA/*/i2MOErA/*/i2MOErT/*/i2MOErG/*/
i2MOErC/*/i2MOErC/*/i2MOErA/*/i2MOErG/*/i2MOErT/*/32MOErA;
(SEQ ID NO: 46)
52MOErC/*/i2MOErT/*/i2MOErC/*/i2MOErA/*/i2MOErA/*/i2MOErG/*/i2MOErC/*/
i2MOErC/*/i2MOErT/*/i2MOErT/*/i2MOErA/*/i2MOErT/*/i2MOErT/*/i2MOErA/*/
i2MOErC/*/i2MOErA/*/i2MOErA/*/i2MOErT/*/i2MOErG/*/32MOErC;
(SEQ ID NO: 47)
52MOErC/*/i2MOErC/*/i2MOErC/*/i2MOErT/*/i2MOErC/*/i2MOErA/*/i2MOErA/*/
i2MOErG/*/i2MOErC/*/i2MOErC/*/i2MOErT/*/i2MOErT/*/i2MOErA/*/i2MOErT/*/
i2MOErT/*/i2MOErA/*/i2MOErC/*/i2MOErA/*/i2MOErA/*/32MOErT;
or
(SEQ ID NO: 48)
52MOErC/*/i2MOErC/*/i2MOErC/*/i2MOErT/*/32MOErC/*/i2MOErT/*/i2MOErC/*/
i2MOErC/*/i2MOErC/*/i2MOErT/*/32MOErC/*/32MOErT,
wherein “52MOEr” indicates a 5′-terminal 2′-O-methoxyethyl modification, “i2MOEr” indicates
an internal 2′-O-methoxyethyl modification, “32MOEr” indicates a 3′-terminal 2′-O-
methoxyethyl modification, and an asterisk indicates the presence of a phosphorothioate
internucleoside linkage.

In some embodiments, the target RNA transcript includes all or a portion of

(SEQ ID NO: 12)
GGGAGAGGGAGAGGGAGAGCTATTTCATTTTTTTTTTTTACACATAAGATGTGG
AAAAAAAATGTACTGGCATTGTAATAAGGCTTGAGGGAGGCACATCTCACACA
TGAACGTGAAAACCCAATGTCGTCACACTTACGTCATCAAAAGC;
(SEQ ID NO: 13)
GGGAGAGGGAGAGGGAGAGGGAGAGCTATTTCATTTTTTTTTTTTCCACATCA
GATGTGGAAAAAAAATGTACTGGCATTGTAATAAGGCTTGAGGGAGGCACATC
TCACACATGAACGTGAAAACAGATCGGAAGAGCGTCGGGTAGGGA;
(SEQ ID NO: 14)
GGGAGAGGGAGAGGGAGAGCTATTTCATTTTTTTTTTTTCCACATCAGATGTG
GAAAAAAAATGTACTGGCATTGTAATAAGGCTTGAGGGAGGCACAGCTCACA
CATGAACGTGAAAACCCAATGTCGTCACACGTACGGCATCCTAAG;
(SEQ ID NO: 15)
GGGAGAGGGAGAGGGAGAGCTATTTCATTTTTTTTTTTTACACATCAGATGTG
GAAAAAAAATTTACTGGCATTGTAATAAGGCTTGAGGGAGGCACATCTCACAC
ATGAACGTGAAAACCCAAAGTAGTCACACTTACGTCATCCTAAGC;
(SEQ ID NO: 16)
GGGAGAGGGAGAGCTATTTCATTTTTTTTTTTTCCACATCAGATGTGGAAAAA
AAATGTACTGGCATTGTAATAAGGCTTGAGGGAGGCACATCTCACACATGAAC
GTGAAAACCCAATGTCGTCACACTTACGTCATCATAAGCTTATGA;
(SEQ ID NO: 17)
GGAGAGGGAGAGCTATTTCATTTTTTTTTTTTCCACATCATATGTGGAAAAAAA
ATGTACTGGCATTGTAATAAGGCTTGAGGGAGGCACATCTCACACATGAACGT
GAAAAACCAATGTCGTCACACTTACGTAATAATAAGAGTATGTA;
(SEQ ID NO: 18)
GGGAGAGGGAGAGGGAGAGCTATTTCATTTTTTTTTTTTCCACATCAGATGTG
GAAAAAAAATGTACTGGCATTGTAATAAGGCTTGAGGGAGGCACATCTCACAC
ATGAACGTGAAAACCCAATGTCGTCACACTTACGTCATCCTAAGC;
(SEQ ID NO: 19)
GCTATTTCATTTTTTTTTTTTTTCCACATCAGTTGTGGAAAAAAAATGTACTGGC
ATTGTAATAAGGCTTGAGGGAGGCACATCTCACACATGAACGTGAAAACCCAA
TGTCGTCACACTTACGTCATCATAAGCTTATGAACTACAAAAA;
(SEQ ID NO: 20)
GAGGAGAGGGAGAGGGAGAGGGAGAGGGAGAGGGAGAGGGAGAGGGAGA
GCTATTTCATTTTTTTTTTTTCCACATCAGATGTGGAAAAAAAATGTACTGGCAT
TGTAAAAAGGCTTGAGGGAGGCACATCTCACACATGAACGTGAAAA;
(SEQ ID NO: 21)
AAGAGAGGGAGAGGGAGAGGGAGAGGGAGAGGGAGAGGGAGAGGGAGAG
CTATTTCATTTTTTTTTTTTCCACATCAGATGTGGAAAAAAAATGTACTGGCATT
GTAATAAGGCTTGAGGGAGGCACATCTCACACATGAACGTGAAAACC;
(SEQ ID NO: 22)
GAGAGGGAGAGGGAGAGGGAGAGGGAGAGCTATTTCATTTTTTTTTTTTCCAC
ATCAGATGTGGAAAAAAAATGTACTGGCATTGTAATAAGGCTTGAGGGAGGCA
CATCTCACACATGAACGTGAAAACCCCATGTCGTCACACTTACGG;
(SEQ ID NO:23)
GATGGGGAGAGCTATTTCATTTTTTTTTTTTCCCCATCAGATGTGGAAAAAAAA
TGTACTGGCATTGTAATAAGGCTTGAGGGAGGCACATCTCACACATGAACGTG
AAAACCCAATGTCGTCACACTTACGAGATCGGAAGAGCGTCGGG;
or
(SEQ ID NO: 24)
GATGGGGAGAGCTATTTCATTTTTTTTTTTTCCCCATCAGATGTGGAAAAAAAA
TGTACTGGCATTGTAATAAGGCTTGAGGGAGGCACATCTCACACATGAACGTG
AAAACCCAATGTCGTCACACTTACGAGATCGGAAGAGCGTCGGG.

In some embodiments, the target RNA transcript includes all or a portion of the reverse complement of any of SEQ ID NO: 12; SEQ ID NO:13; SEQ ID NO: 14; SEQ ID NO: 15; SEQ ID NO:16; SEQ ID NO:17; SEQ ID NO:18; SEQ ID NO:19; SEQ ID NO: 20; SEQ ID NO:21; SEQ ID NO:22; SEQ ID NO:23; or SEQ ID NO:24.

In some embodiments, the pharmaceutical composition comprising any of SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11 SEQ ID NO:25, or SEQ ID NO: 26 hybridizes to a target RNA transcript comprising any of SEQ ID NO: 12; SEQ ID NO: 13; SEQ ID NO:14; SEQ ID NO:15; SEQ ID NO:16; SEQ ID NO:17; SEQ ID NO: 18; SEQ ID NO: 19; SEQ ID NO:20; SEQ ID NO:21; SEQ ID NO:22; SEQ ID NO: 23; or SEQ ID NO:24. In some embodiments, the pharmaceutical composition comprising any of SEQ ID NO:2, SEQ ID NO:3, SEQ ID NO:4, SEQ ID NO:5, SEQ ID NO: 6, SEQ ID NO:7, SEQ ID NO:8, SEQ ID NO:9, SEQ ID NO: 10, SEQ ID NO:11, SEQ ID NO:25, or SEQ ID NO:26 promotes the degradation of a target RNA transcript comprising any of SEQ ID NO: 12; SEQ ID NO: 13; SEQ ID NO:14; SEQ ID NO: 15; SEQ ID NO:16; SEQ ID NO: 17; SEQ ID NO:18; SEQ ID NO: 19; SEQ ID NO:20; SEQ ID NO: 21; SEQ ID NO:22; SEQ ID NO:23; or SEQ ID NO:24. In some embodiments the pharmaceutical composition comprising any of SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 10, SEQ ID NO:11, SEQ ID NO:25, or SEQ ID NO:26 sterically inhibits the translation of a target RNA transcript comprising any of SEQ ID NO: 12; SEQ ID NO:13; SEQ ID NO: 14; SEQ ID NO:15; SEQ ID NO: 16; SEQ ID NO:17; SEQ ID NO:18; SEQ ID NO: 19; SEQ ID NO:20; SEQ ID NO:21; SEQ ID NO:22; SEQ ID NO:23; or SEQ ID NO: 24. In some embodiments the pharmaceutical composition comprising any of SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:25, or SEQ ID NO: 26 is therapeutically effective for treating a disease caused by a target RNA transcript comprising any of SEQ ID NO: 12; SEQ ID NO: 13; SEQ ID NO: 14; SEQ ID NO: 15; SEQ ID NO:16; SEQ ID NO: 17; SEQ ID NO:18; SEQ ID NO:19; SEQ ID NO: 20; SEQ ID NO:21; SEQ ID NO:22; SEQ ID NO:23; or SEQ ID NO:24. In some embodiments, the pharmaceutical composition comprising any of SEQ ID NO:2, SEQ ID NO: 3, SEQ ID NO:4, SEQ ID NO:5, SEQ ID NO:6, SEQ ID NO:7, SEQ ID NO:8, SEQ ID NO: 9, SEQ ID NO: 10, SEQ ID NO: 11, SEQ ID NO:25, or SEQ ID NO:26 sterically inhibits aberrant intron retention within a target RNA transcript comprising any of SEQ ID NO: 12; SEQ ID NO:13; SEQ ID NO:14; SEQ ID NO:15; SEQ ID NO:16; SEQ ID NO: 17; SEQ ID NO:18; SEQ ID NO:19; SEQ ID NO:20; SEQ ID NO:21; SEQ ID NO: 22; SEQ ID NO:23; or SEQ ID NO:24. In some embodiments, the pharmaceutical composition comprising any of SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO: 10, SEQ ID NO: 11, SEQ ID NO:25, or SEQ ID NO:26 sterically inhibits R-loop formation within a target RNA transcript comprising any of SEQ ID NO:12; SEQ ID NO:13; SEQ ID NO: 14; SEQ ID NO: 15; SEQ ID NO: 16; SEQ ID NO: 17; SEQ ID NO: 18; SEQ ID NO: 19; SEQ ID NO:20; SEQ ID NO:21; SEQ ID NO:22; SEQ ID NO:23; or SEQ ID NO: 24. In some embodiments, the pharmaceutical composition comprising any of SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:25, or SEQ ID NO: 26 sterically inhibits binding of RNA binding proteins and/or small RNAs within a target RNA transcript comprising any of SEQ ID NO: 12; SEQ ID NO:13; SEQ ID NO: 14; SEQ ID NO: 15; SEQ ID NO: 16; SEQ ID NO:17; SEQ ID NO:18; SEQ ID NO: 19; SEQ ID NO:20; SEQ ID NO:21; SEQ ID NO:22; SEQ ID NO:23; or SEQ ID NO: 24.

### Methods of Treatment

Also disclosed herein are methods of treating a disease in a human subject. In some embodiments, the method of treating a disease in a human subject is a method of treating XDP. In some embodiments, the method of treating a disease in a human subject is a method of treating XDP using antisense oligonucleotides.

The methods of treatment described herein can include the administration of pharmaceutical compositions and formulations comprising inhibitory nucleic acid sequences designed to target a disease-associated RNA transcript, e.g., a transcript transcribed from a locus within the TAF1 gene.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions disclosed herein include those suitable for parenteral administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form can vary depending upon the host being treated, the particular mode of administration. The amount of active ingredient which can be combined with a carrier material to produce a single dosage form will generally be that amount of the compound which produces a therapeutic effect, e.g., degradation or steric blockage of a target RNA transcript and/or reduction in a symptom of a disease in a patient. In some embodiments, the RNA transcript is transcribed from a locus within the TAF1 gene and the disease is XDP.

In some embodiments, the compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., extent of relieving dystonia-related symptoms), and the like. The formulations should provide a sufficient quantity of active agent to effectively treat, prevent or ameliorate conditions, diseases or symptoms.

### Organoid Compositions

Also disclosed herein are compositions directed organoids. In some embodiments, the organoids are generated from cells derived from a human. In some embodiments, the organoids are derived from induced pluripotent stem cells (iPSCs). In some embodiments the organoids are striatal organoids. In some embodiments the organoids are used to study the molecular mechanisms of a human disease. In some embodiments the organoids are used to study the molecular mechanisms of XDP or to test the efficacy of compositions and methods for treating XDP.

In some embodiments the organoid compositions include a high abundance of neural progenitor cells. The neural progenitor cells can be shown to express various characteristic biomarkers including Ki-67, SOX2, NESTIN proteins. The neural progenitor cells may self-organize into polarized neuroepithelium-like loops within the organoid structures. The neural progenitor cells may express the forebrain development marker FOXG1 and striatal commitment marker MEIS2. In some embodiments the organoid compositions include mature neuronal cells that express MAP2, DARPP32, GABA, or CTIP2. The neuronal cells of striatal organoids may express markers of two striosomal compartments, matrix (CALB1) and striosome (CALB2). In some embodiments, the organoid compositions include medium spiny neurons (MSNs), a specialized type of GABAergic inhibitory cell representing approximately 95% of neurons within the human striatum. In some embodiments, neuronal cells may represent about 10%, 15%, 20%, 25%, 30%, 35%, 40%, 45%, 50%, 55%, or about 60% of cells within the organoid composition, or value therebetween.

In some embodiments, the organoids are generated from iPSCs derived from cells collected from a human subject. The human subject can be a patient harboring a genetic disease, an asymptomatic carrier of a genetic disease, an unaffected relative of a patient harboring a genetic disease, or an unaffected healthy “control” individual. In some embodiments, the organoids are generated from iPSCs derived from cells collected from a patient having XDP. In some embodiments, the striosome marker CALB2 is downregulated in striatal organoids generated from cells collected from an XDP patient relative to in striatal organoids generated from cells collected from an unaffected control. In some embodiments, CALB2 is downregulated in female mutant carrier-derived striatal organoids.

In some embodiments, cells within the organoid composition express the TAF1 protein. TAF1 protein expression may be decreased in mature striatal organoids (greater than 90 days old) relative to less mature striatal organoids (less than 30 days old). Striatal organoids may comprise RNA transcripts transcribed from the TAF1 gene. The RNA transcripts transcribed from the TAF1 gene may comprise SVA-derived polynucleotide sequence. The RNA transcripts transcribed from the TAF1 gene may be aberrantly spliced due to the presence of an SVA insertion in intron 32 of the TAF1 gene. Transcription of the RNA transcribed from the TAF1 gene may be initiated from the SVA sequence.

In some embodiments, cells of the organoid composition can include the presence of RNA transcripts comprising all or a portion of any of SEQ ID NO: 12; SEQ ID NO:13; SEQ ID NO: 14; SEQ ID NO: 15; SEQ ID NO:16; SEQ ID NO:17; SEQ ID NO:18; SEQ ID NO: 19; SEQ ID NO:20; SEQ ID NO:21; SEQ ID NO:22; SEQ ID NO:23; or SEQ ID NO: 24. In some embodiments, cells of the organoid composition can include the presence of RNA transcripts comprising all or a portion of the reverse complement of any of SEQ ID NO: 12; SEQ ID NO:13; SEQ ID NO:14; SEQ ID NO:15; SEQ ID NO:16; SEQ ID NO: 17; SEQ ID NO:18; SEQ ID NO:19; SEQ ID NO:20; SEQ ID NO:21; SEQ ID NO: 22; SEQ ID NO:23; or SEQ ID NO:24.

In some embodiments, cells of the organoid composition can include the presence of RNA transcripts that include variable numbers of the hexanucleotide repeat CCCTCT (SEQ ID NO:1). For striatal organoids generated from cells collected from an XDP patient, SEQ ID NO: 1 may be present as tandem repeats numbering from about 30 tandem repeats of SEQ ID NO:1 to about 60 tandem repeats of SEQ ID NO:1. For striatal organoids generated from cells collected from an XDP patient, the number of tandem repeats of SEQ ID NO:1 in RNA transcripts within some cells of the organoid may increase as the striatal organoid matures, and the number of cells within the striatal organoid harboring expanded tandem repeats of SEQ ID NO:1 may increase as the striatal organoid matures.

### Methods of Making Organoids

In some embodiments the organoids are generated from iPSCs derived from cells collected from a human subject. The human subject can be a patient harboring a genetic disease, an asymptomatic carrier of a genetic disease, an unaffected relative of a patient harboring a genetic disease, or an unaffected healthy “control” individual. In some embodiments, the disease is XDP. In some embodiments the cells collected from the human subject are fibroblasts. The fibroblasts may be reprogrammed by methods well known in the art to become de-differentiated iPSCs.

In some embodiments, in the process of generating striatal organoids, the iPSCs are grown in DMEM/F12 medium plus Glutamax/N2/B27. In some embodiments, the cells are grown in NeuroBasal medium plus Glutamax and B27. In some embodiments, the cells are grown in BrainPhys medium plus Glutamax. In some embodiments, the cell growth medium is supplemented with Y-27632. In some embodiments, the cell growth medium is supplemented with LDN. In some embodiments the cell growth medium is supplemented with SB. In some embodiments the cell growth medium is supplemented with BDNF, GDNF, IGF-1, and/or dbc-AMP. In some embodiments, the iPSCs are embedded in matrigel droplets to establish organotypic culture for the generation of striatal organoids. In some embodiments, striatal organoids are collected for phenotypic, molecular, and functional characterization.

In some embodiments, after the formation of iPSCs derived from patient cells or unaffected healthy control cells, the iPSCs are contacted with a DKK-1 and Purmorphamine pathway activator for a period of time sufficient for the cells to differentiate into ventral telencephalic progenitors, neurons and glia. Next, the cells are contacted with SB 431542 and LDN-193189 for a period of time sufficient for the cells to form neuroepithelium. Subsequently, the cells are grown in contact with BDNF, GDNF, IGF-1, and dibutyryl-CAMP for a period of time sufficient to promote ventral telencephalic progenitors, neurons, and glia.

### EXAMPLES

### Example 1—Striatal Organoids can be Generated From Patient-Derived iPSCs

To generate striatal organoids from iPSCs, fibroblasts were collected from female XDP-carriers, unaffected male controls, and XDP patients. Fibroblasts were re-programmed and de-differentiated to form iPSCs according to standard methods. Briefly, fibroblasts between passages 5 and 7 were reprogrammed with episomal vectors. Plasmids pCXLE-hOCT3/4-shp53-F (Addgene #27077), pCXLE-hSK (#27078), and pCXLE-hUL (#27080) were transfected into fibroblasts using 4D-Nucleofector system with P2 Primary Cell 4D-Nucleofector X Kit (Lonza; program DT-130). Three to five weeks after reprogramming, single colonies were picked and expanded on SNL76/7 feeder cells (ATCC, SCRC-1049) treated with mitomycin C (Sigma Aldrich) in 20% KSR medium. iPS cells were then transferred to feeder-free culture on Cultrex Reduced Growth Factor Basement Membrane Matrix (Trevigen)-coated plates in StemFlex medium (Gibco). Cells were passaged every 4-6 days with Versene solution (Gibco). A control iPS cell line (MIN09i-33114.C) was obtained from WiCell and maintained in the same way. All lines underwent extensive characterization for identity, pluripotency, and exogenous reprogramming factor expression.

To generate striatal organoids, iPS cells cultured on Cultrex-coated 6-well plates in StemFlex medium were treated with 10 μM Y-27632 (BioGems) at 37° C. overnight when they reach 50-60% confluency. Cells were then incubated with StemPro Accutase (Gibco) at 37° C. for 7 min and dissociated into single cells. Dissociated iPS cells were plated on 96-well V-bottom plates (S-bio) at 9,000 cells/150 μL per well in StemFlex medium with 30 μM Y-27632 and incubated at 37° C. for 2 days. The StemFlex medium was gradually switched to DFN2 consisting of DMEM/F12+GlutaMAX (Gibco) supplemented with 1×N-2 supplement (Gibco), 1×non-essential amino acid (NEAA, Gibco), 100 μM 2-melcaptoethanol (2-ME, Gibco) and 1×Antibiotic-Antimycotic (Gibco) from day 0. From day 6, the medium was gradually switched to DFN2B27 consisting of DMEM/F12+GlutaMAX supplemented with 1×N-2 supplement, 1×NeuroCult SM1 without vitamin A (StemCell Technologies) and 1×Antibiotic-Antimycotic. On day 12, each embryoid body (EB) was embedded in a droplet of Matrigel Basement Membrane Matrix Growth Factor Reduced (Corning) and transferred to a 60-mm culture dish in DFN2B27. The medium was replaced on day 14. On day 16, after 4 days in static culture, the medium was switched to NBB27 consisting of Neurobasal medium (Gibco) supplemented with 1×GlutaMAX (Gibco), 1×NeuroCult SM1 (StemCell Technologies) and 1×Antibiotic-Antimycotic, and the cultures were then agitated in an incubator shaker (Eppendorf, New Brunswick S41i) at 80 r.p.m. The medium was supplemented with 10 μM SB 431542 (BioGems) from day 0 to day 6, 100 nM LDN-193189 (BioGems) from day 0 to day 12, 100 ng/mL human DKK-1 (PeproTech) and 0.65 μM Purmorphamine (BioGems) from day 6 to day 30, and 20 ng/mL human BDNF (PeproTech), 20 ng/mL human GDNF (PeproTech), 10 ng/ml human IGF-1 (PeproTech) and 100 μM Dibutyryl-cAMP (BioGems) from day 30 to day 60. The cells were fed every 3 days from day 0 to day 60 except for day 12 and day 14.From day 60, Dibutyryl-cAMP was withdrawn from the medium and the cells were fed every 3-4 days.

In another modification of the protocol, EBs formed by feeder-free iPS cells (grown in Stem Flex or TSER) were generated by dissociating colonies via collagenase and grown for 1 day in pluripotent media. Medium was switched to DFN2 consisting of DMEM/F12+GlutaMAX (Gibco) supplemented with 1×N-2 supplement (Gibco), 1×non-essential amino acid (NEAA, Gibco), 100 μM 2-melcaptoethanol (2-ME, Gibco) and 1×Antibiotic-Antimycotic (Gibco) from day 0. From day 6, the medium was gradually switched to DFN2B27 consisting of DMEM/F12+GlutaMAX supplemented with 1×N-2 supplement, 1×NeuroCult SM1 without vitamin A (StemCell Technologies) and 1×Antibiotic-Antimycotic. On day 12, each embryoid body (EB) was embedded in a droplet of Matrigel Basement Membrane Matrix Growth Factor Reduced (Corning) and transferred to a 60-mm culture dish in DFN2B27. The medium was replaced on day 14. In another modification, embryoid bodies were not embedded in the droplet of Matrigel. On day 16, after 4 days in static culture, the medium was switched to NBB27 consisting of Neurobasal medium (Gibco) supplemented with 1×GlutaMAX (Gibco), 1×NeuroCult SM1 (StemCell Technologies) and 1×Antibiotic-Antimycotic, and the cultures were then agitated in an incubator shaker (Eppendorf, New Brunswick S41i) at 80 r.p.m. The medium was supplemented with 10 μM SB 431542 (BioGems) from day 0 to day 6, 100 nM LDN-193189 (BioGems) from day 0 to day 12, 100 ng/ml human DKK-1 (PeproTech) and 0.65 μM Purmorphamine (BioGems) from day 6 to day 30, and 20 ng/ml human BDNF (PeproTech), 20 ng/ml human GDNF (PeproTech), 10 ng/ml human IGF-1 (PeproTech) and 100 μM Dibutyryl-cAMP (BioGems) from day 30 to day 60. The cells were fed every 3 days from day 0 to day 60 except for day 12 and day 14. From day 60, Dibutyryl-cAMP was withdrawn from the medium and the cells were fed every 3-4 days.

Beginning on day 12, striatal organoids derived from control iPS cell lines showed suppressed expression of pluripotent stem cell marker, OCT4/POU5F1 and upregulation of genes that represent induction of neural fate and differentiation of ventral and dorsal forebrain compared to undifferentiated iPS cells. By day 37, striatal organoids exhibited rosette-like structure consisting of ventrally fated neuronal progenitor cells (NPCs) expressing MEIS2 and Ki67-positive proliferative cells around ventricles labeled with ZO-1. Intermediate progenitor cells (IPCs)/immature neurons expressing PSA-NCAM and CTIP2 were detected outside the structure. GABAergic neurons and cells fated to MSNs expressing FOXP1, MEIS2, CTIP2 or DARPP32 appeared in VTOs by day 70, and Calretinin (CALR)-expressing GABAergic interneurons in addition to MSNs positive for DARPP32 or Calbindin (CALB) were observed by day 150.

### Example 2—Striatal Organoids Express Neuronal Markers

To investigate the expression of tissue-specific neuronal markers in striatal organoids generated from XDP patient cells and unaffected healthy control cells, immunostaining for neuronal markers was performed. Organoids were fixed with 4% PFA in PBS overnight at 4 degrees C. After washing with PBS, organoids were placed in serial dilutions of PBS-buffered sucrose (10, 20, and 30%, in sequence) 4 degrees C. Each solution was replaced every day. The dehydrated organoids were maintained in 30% sucrose solution at 4 degrees C. until embedding with OCT compound (Sakura Finetek). One day before cryosectioning, fixed organoids were placed in a 1:2 mixture of 30% sucrose solution and OCT compound and left overnight at 4 degrees C. Then, organoids were embedded in a 1:2 mixture of 30% sucrose solution and OCT compound, frozen immediately in dry ice/acetone and cryosectioned at 10 μm. Tissue sections were subjected to additional fixation with 4% PFA in PBS for 3 min at room temperature, following which they were permeabilized and blocked with 10% normal donkey serum in PBS containing 0.3% Triton X-100 for 30 min at room temperature. After washing with 5% serum in PBS containing 0.01% Tween-20, sections were incubated with primary antibodies at 4 degrees C. overnight and with secondary antibodies at room temperature for 90 min.

As shown in FIG. 4A, striatal organoids after 30 days of organotypic culture showed proliferative neural progenitor cells self-organizing into polarized neuroepithelium-like loop structures that stained positive for Ki67, SOX2, and NESTIN by immunofluorescence. After 30 days the organoids also showed abundant expression of forebrain development marker FOXG1 and striatal commitment marker MEIS2 after 30 days of culture.

As shown in FIG. 4B, striatal organoids after 120 days of organotypic culture, a high proportion of mature neurons (positive for MAP2 expression) expressed markers of medium spiny neurons, such as DARPP32, GABA, and CTIP2. Additionally, neuronal cells of the striatal organoids after 120 days of culturing expressed markers of two striosomal compartments, matrix (CALB1) and striosome (CALB2).

### Example 3—XDP Patient-Derived Striatal Organoids Express Alternatively Spliced TAF1-32i RNA Transcripts

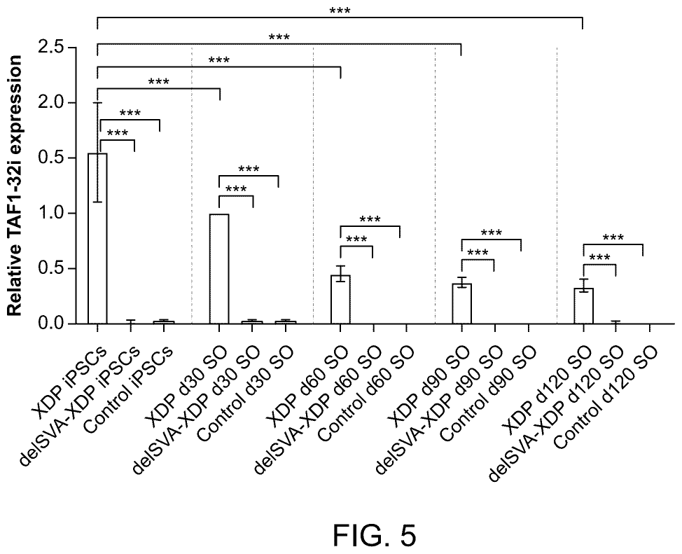

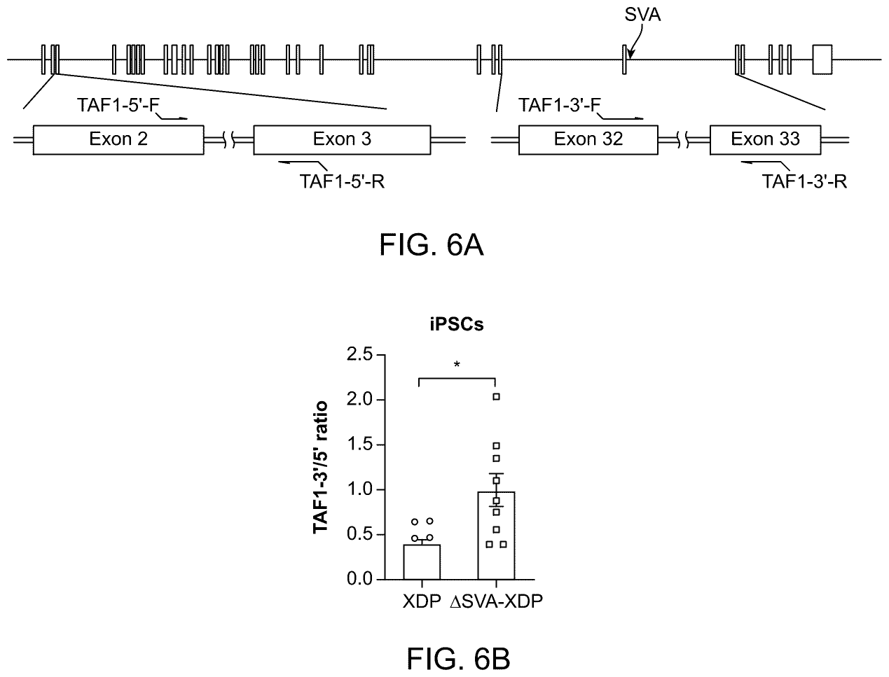

To detect and quantify the abundance of alternatively spliced TAF1-32i RNA transcripts in the striatal organoids, qPCR was performed on cells from the organoids. Prior to RNA extraction, organoids were minced with a blade. Total cellular RNA was extracted from small pieces of organoids using TRIzol Reagent (Invitrogen) and a Direct-zol RNA MiniPrep kit (Zymo Research), in accordance with the manufacturer's instructions. cDNA was prepared by reverse transcription using the SuperScript IV VILO Master Mix (Invitrogen). Real-time qPCR was carried out using QuantiTect SYBR Green PCR Kit (Qiagen) on QuantStudio 3 Real-Time PCR System (Applied Biosystems). Primers were designed directed to the TAF1-32i RNA transcript (see FIG. 6A) and the relative TAF1-32i expression was quantified for iPSCs and iPSC-derived striatal organoids generated from XDP patients, delSVA-XDP cells, and unaffected healthy controls (see FIG. 5). Also, the TAF1-3′/5′ ratio was quantified for XDP iPSCs and delSVA-XDP iPSCs (see FIG. 6B). TAF1 RNA dysregulation can serve as a disease-specific biomarker, with XDP samples exhibiting a decreased TAF1-3′/5′ ratio relative to delSVA-XDP samples where the SVA insertion had been excised from the TAI/locus with CRISPR/Cas9.

### Example 4—XDP Patient-Derived Striatal Organoids Express SVA-Derived RNA Transcripts

To detect and quantify the abundance of SVA-derived RNA transcripts in the striatal organoids, total RNA sequencing and qPCR was performed on cells from the organoids. Prior to RNA extraction, organoids were minced with a blade. Total cellular RNA was extracted from small pieces of organoids using TRIzol Reagent (Invitrogen) and a Direct-zol RNA MiniPrep kit (Zymo Research), in accordance with the manufacturer's instructions and subsequent DNA removal with TurboDNAse. cDNA was prepared by reverse transcription using SuperScript III First-strand synthesis SuperMix (Invitrogen). Real-Time PCR was carried out using TaqMan Fast Advanced Master Mix (Applied biosystems) on QuantStudio3 Real-Time PCR System to detect the TAF1-SVA derived transcript and normalized to GAPDH expression. Primers and probe were designed directed to the TAF1-SVA RNA transcript (see FIG. 7A) and the relative TAF1-SVA expression was quantified for iPSCs and iPSC-derived striatal organoids generated from XDP patients and unaffected healthy controls (see FIG. 7B).

### Example 5—Antisense Oligonucleotides Targeting SVA-Derived RNA Transcripts

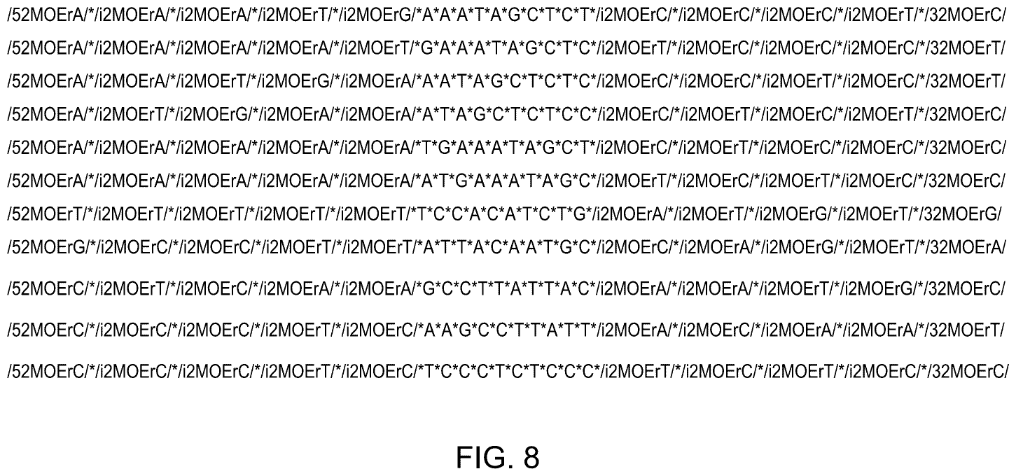

Antisense oligonucleotides were designed to target the SVA-derived RNA transcripts of the TAF1 gene detected in iPSCs and striatal organoids generated from XDP patient cells. For example, FIG. 8 shows ten modified ASOs that were designed to target SVA-derived TAF1 RNA transcripts. Each of the modified ASOs is approximately 15-25 in length and is designed to specifically hybridize to the SVA-derived RNA transcript. As shown in FIG. 8, an asterisk between each nucleotide base (e.g. A, C, T, G) represents a phosphorothioate bond between the bases. “52MOErN” and “32MOErN” indicate a 2′-O-methoxy-ethyl nucleotide base at the 5-prime and 3-prime ends of the ASO, respectively. “i2MOErN” indicates an internal 2′-O-methoxy-ethyl nucleotide base in the ASO. The ASOs shown in FIG. 8 are gapmers, i.e., short DNA antisense oligonucleotide structures with modified RNA-like segments on both sides of the sequence.

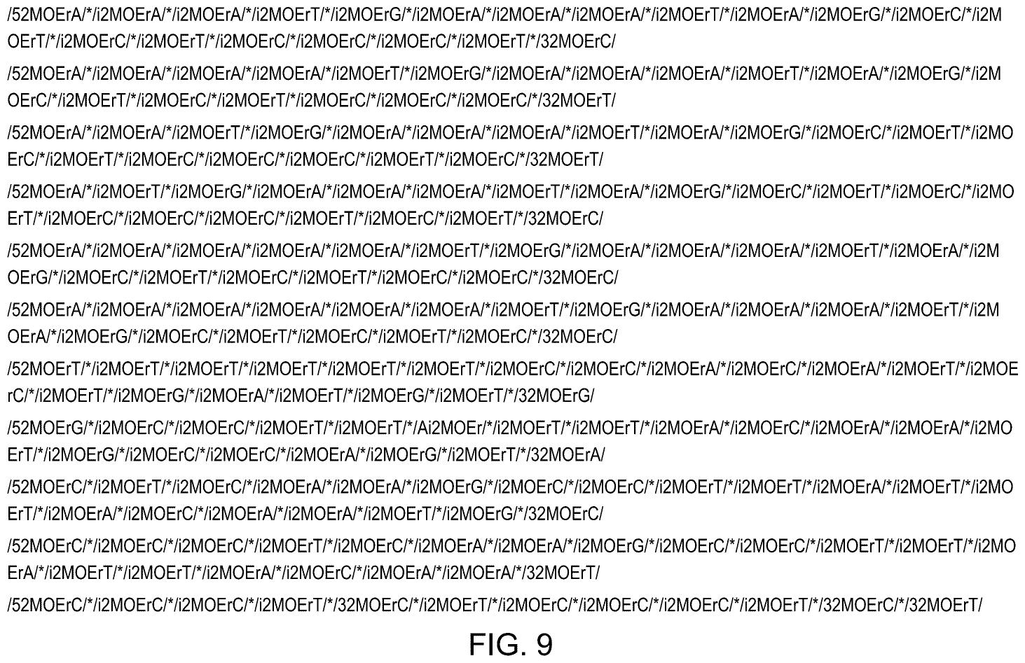

Additionally, ASOs were designed to target the SVA-derived RNA transcripts of the TAF1 gene and modified to perform steric blocking, such that the fully i2MOErN ASOs include phosphorothioate bonds throughout. Ten exemplary ASOs including phosphorothioate bonds throughout are shown in FIG. 9. To inhibit tetraplex formation, portions or all of the guanosine and cytosine residues may be replaced with 7-deazaG, 7-daxaC, or 6-thioG, 6-thioC. ASOs may be shortened or extended.

### Example 6—SVA-Derived Transcripts Induce Neurodegenerative Phenotype in XDP Ventral Forebrain Organoids

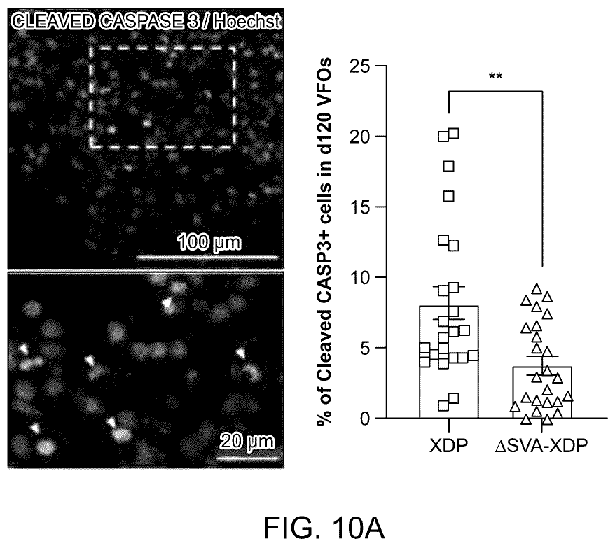

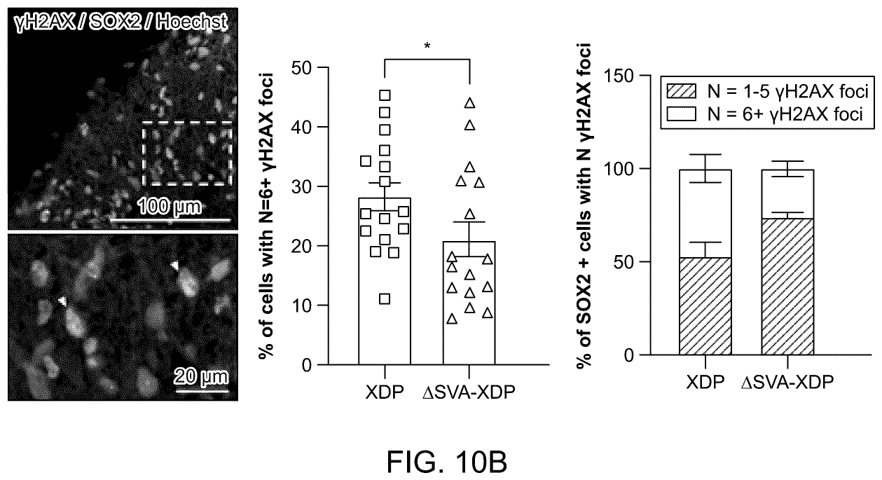

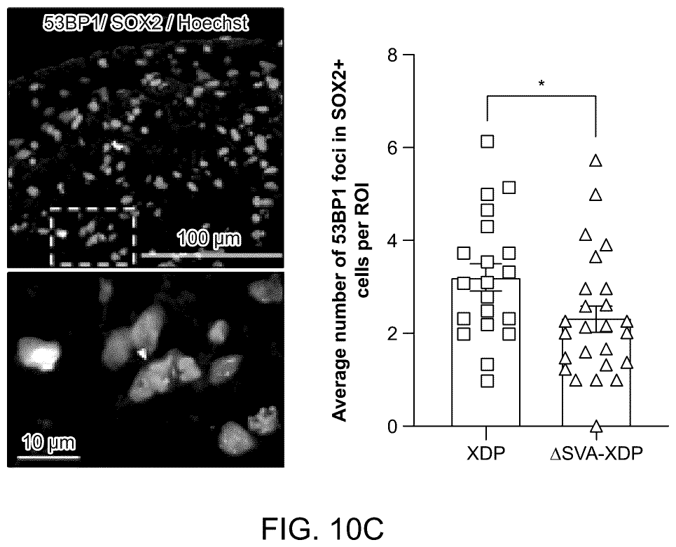

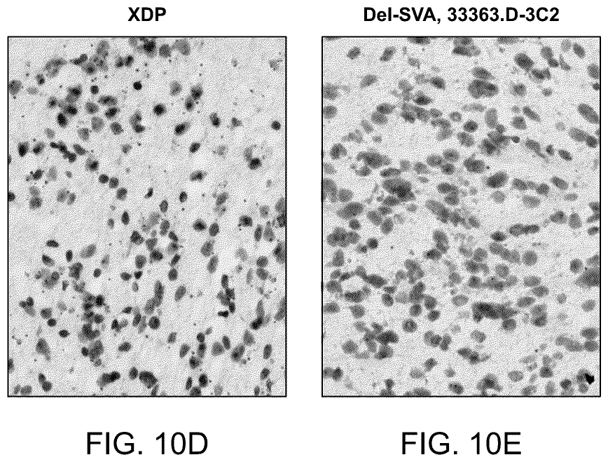

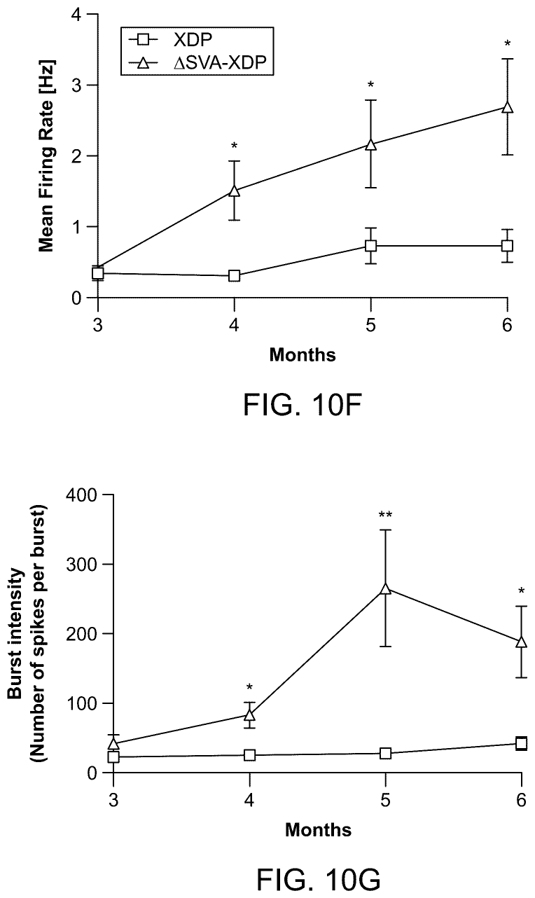

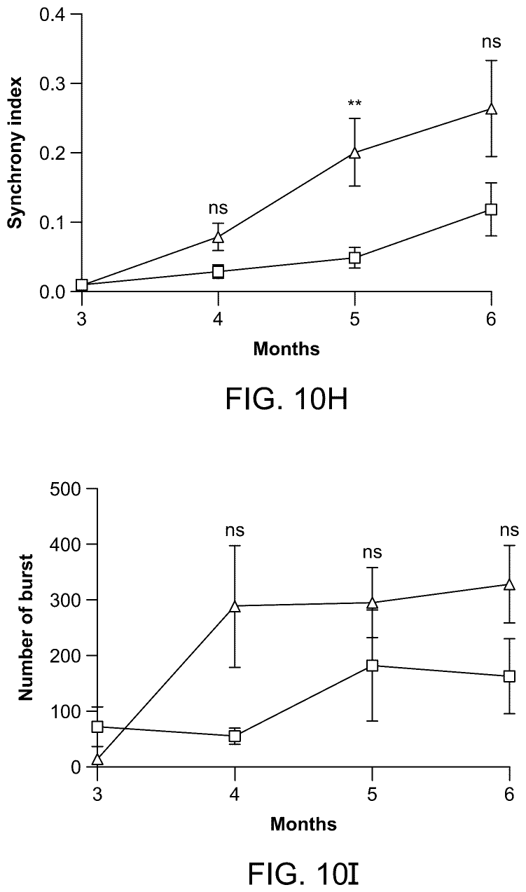

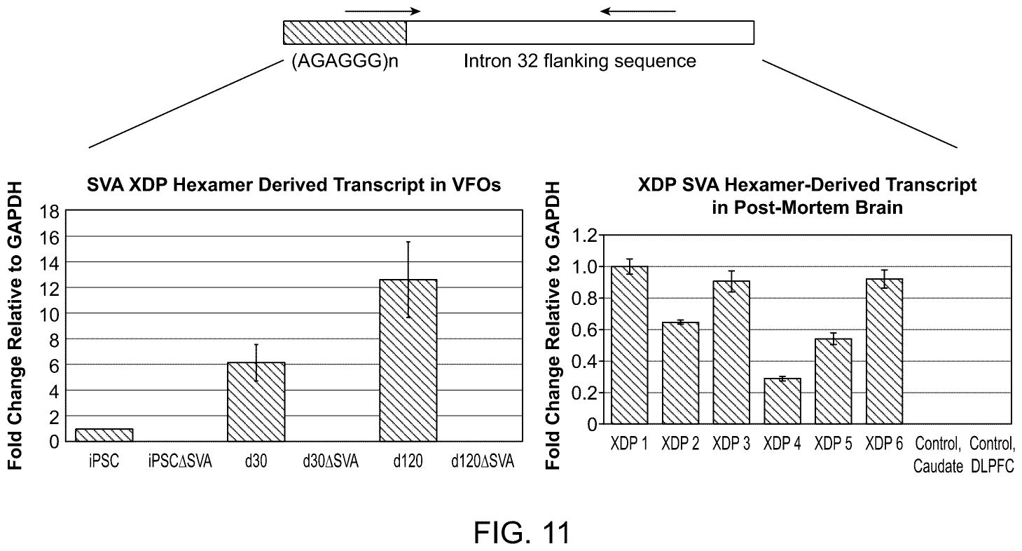

In order to characterize the phenotype induced by TAF1 SVA-derived transcripts in XDP organoids, immunohistochemistry was performed for apoptotic and DNA damage markers and electrophysiological phenotypes were characterized. FIG. 10A shows a fluorescent image and plot of quantification of immunohistochemistry for Cleaved Caspase 3, an apoptotic marker, in 120-day XDP and isogenic control (ΔSVA-XDP) organoids. The XDP organoids show a higher percentage of cleaved CASP3+cells than the isogenic control (ΔSVA-XDP) organoids, demonstrating increased apoptosis in the XDP organoids (** p<0.001, 2-tailed t-test). FIG. 10B shows a fluorescent image and plot of quantification of immunohistochemistry for yH2AX, a DNA damage marker, in 120-day XDP and isogenic control (ΔSVA-XDP) organoids. The XDP organoids show a higher percentage of cells with yH2AX foci than the isogenic control organoids, demonstrating increased DNA damage in the XDP organoids (* p<0.05, 2-tailed t-test). FIG. 10C shows a fluorescent image and plot of quantification of immunohistochemistry for 53BP1, a DNA damage marker, in 120-day XDP and isogenic control (ΔSVA-XDP) organoids. The XDP organoids show a higher percentage of cells with 53BP1 foci than the isogenic control organoids, demonstrating increased DNA damage in the XDP organoids (* p<0.05, 2-tailed t-test).

FIGS. 10D-10E show in situ hybridization of RNA aggregates in 120-day XDP (D) and isogenic control organoids (E). The RNA aggregates are detected specifically in the 120-day XDP organoids.

In order to evaluate the electrophysiological phenotype of XDP organoids expressing the TAF1 SVA-derived transcripts, multielectrode array (MEA) experiments were performed. FIGS. 10F-10I are plots of mean firing rate (F), burst intensity (G), synchrony index (H) and number of bursts (I) as measured by MEA in 120-day XDP and isogenic control (ΔSVA-XDP) organoids. For each measurement, the 120-day XDP organoids exhibited decreased electrophysiological activity compared to the isogenic control (ΔSVA-XDP) organoid, indicating a neurodegenerative phenotype in the 120-day XDP organoids.

### Example 7—SVA-Derived RNA Transcripts Accumulate in XDP Organoids and XDP Patient Postmortem Brain

In order to measure the abundance of TAF1 SVA-derived transcripts in XDP cells and tissues, RT-qPCR was performed in XDP induced pluripotent stem cells (iPSCs), 30-day organoids, 120-organoids, and in postmortem brain tissue collected from several XDP patients. FIG. 11 (top) shows a schematic of the RT-qPCR assay used to measure the abundance of TAF1 SVA-derived transcripts, with the forward primer targeting the SVA repeat and the reverse primer targeting the flanking sequence of TAF1 intron 32.

As shown in FIG. 11 (lower left), the SVA-derived transcript was detected in XDP iPSCs, 30-day organoids, and 120-organoids, but not in isogenic controls. Notably, the SVA-derived transcript abundance is progressively higher in the 30-day and 120-day organoids relative to the iPSCs, indicating that the SVA-derived transcript accumulates in cells.

As shown in FIG. 11 (lower right), the SVA-derived transcript was detected in post-mortem brain tissue collected from six XDP patients. The SVA-derived transcript was not detected in tissue from a healthy control caudate nucleus and a healthy control dorsolateral prefrontal cortex.

### Example 8—Antisense Oligonucleotide Knockdown of SVA-Derived RNA Transcripts in XDP Organoids

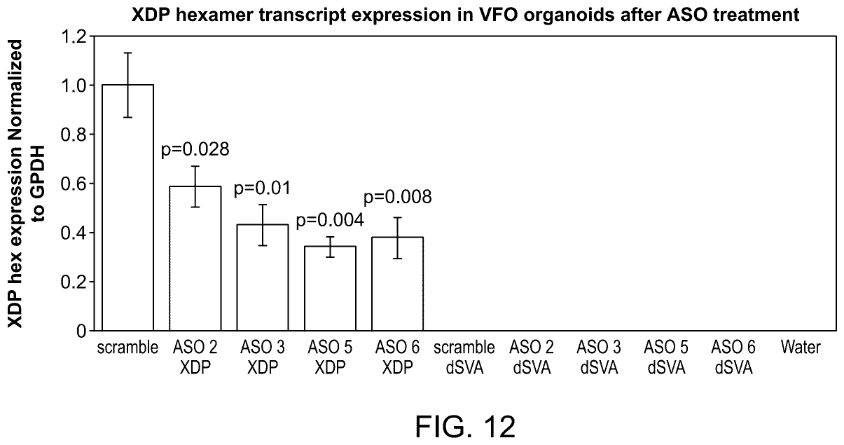

In order to measure the extent of knockdown of SVA-derived RNA transcripts by the ASOs disclosed herein, RT-qPCR quantification of TAF1 XDP SVA-derived transcript expression was performed in mature XDP ventral forebrain organoids treated with the ASOs disclosed herein. FIG. 12 is a plot of TAF1 XDP SVA transcript expression normalized to GAPDH in XDP ventral forebrain organoids. Organoids treated with ASO 2 (SEQ ID NO: 3), ASO 3 (SEQ ID NO: 4), ASO 5 (SEQ ID NO: 6), and ASO 6 (SEQ ID NO: 7) each showed decreased expression of the TAF1 XDP SVA-derived transcript relative to organoids treated with a scrambled ASO as a control.

### Example 9—Hominid-Specific SVA Retrotransposons Contain a Hexamer Tandem Repeat With Antisense Promoter Activity in Human Brain

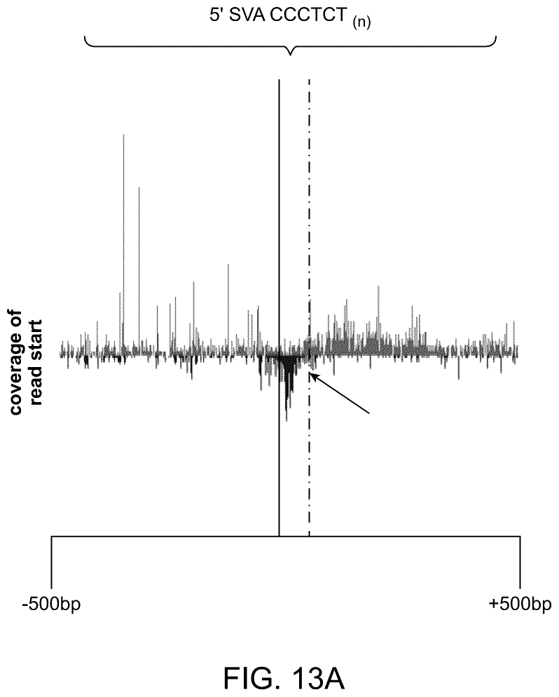

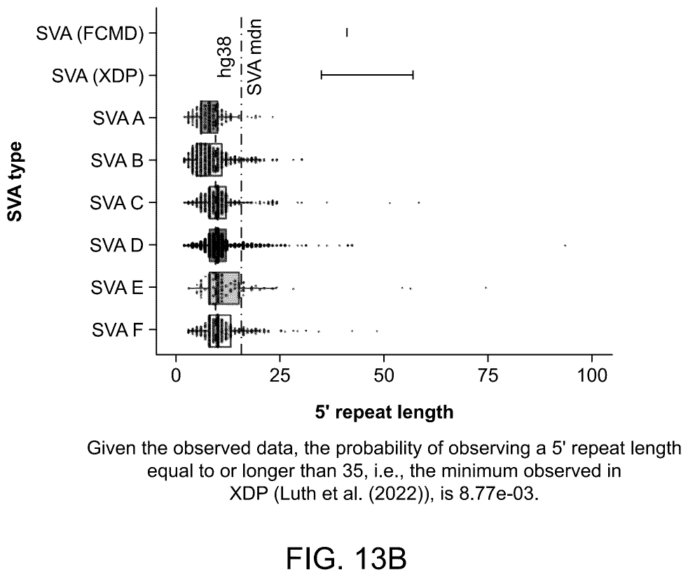

FIG. 13A is a plot of coverage of the first base of long-read RNA-Seq reads (PacBio Iso-Seq) in human brain (ENCODE data, T2T-CHM13). The positive strand is represented above the x-axis, and the negative strand is represented below the x-axis. The solid and dotted lines indicate the boundaries of the 5′ SVA CCCTCT (n) hexamer. The arrow indicates 5′ antisense promoter activity at SVA hexamer repeat loci. These results demonstrate that for SVA loci present in all humans, the 5′ antisense hexamer promoter is active in human brain.

FIG. 13B is a plot of 5′ hexamer repeat length of individual SVA loci by type. Hexamer repeat length range in XDP patients is annotated by a bar on the plot and indicated with an arrow. The majority of SVA loci in the genome contain a hexamer repeat, however, the TAF1 XDP repeat is significantly longer than most other SVA loci in the genome.

A number of embodiments of the invention have been described. Nevertheless, it will be understood that various modifications may be made without departing from the spirit and scope of the invention. Accordingly, other embodiments are within the scope of the following claims.

What is claimed is:

1. A pharmaceutical composition comprising an inhibitory nucleic acid that is complementary to a portion of an RNA transcript derived from a SINE/VNTR/Alu (SVA) element.

2. The composition of claim 1, wherein the RNA transcript comprises a hexanucleotide repeat.

3. The composition of claim 2, wherein the hexanucleotide repeat comprises, or is complementary to, the polynucleotide sequence CCCTCT (SEQ ID NO:1).

4. The composition of claim 1, wherein the RNA transcript comprises a polynucleotide sequence derived from the SVA element and a polynucleotide sequence derived from a mammalian genome.

5. The composition of claim 4, wherein the polynucleotide sequence derived from the mammalian genome maps to a protein-coding gene.

6. The composition of claim 5, wherein the polynucleotide sequence derived from the mammalian genome maps to an intron of the protein-coding gene.

7. The composition claim 5, wherein the protein-coding gene is TAF1.

8. The composition claim 4, wherein the mammalian genome is a human genome.

9. The composition of claim 1, wherein the inhibitory nucleic acid is a double stranded RNA species.

10. The composition of claim 1, wherein the inhibitory nucleic acid is a small interfering RNA (siRNA) species.

11. The composition of claim 1, wherein the inhibitory nucleic acid is a morpholino oligomer.

12. The composition of claim 1, wherein the inhibitory nucleic acid is an antisense oligonucleotide.

13. The composition of claim 12, wherein the antisense oligonucleotide is complementary to at least a portion of the 5′-untranslated region (5′UTR) of the RNA transcript.

14. The composition of claim 12, wherein the antisense oligonucleotide comprises a sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:25, and SEQ ID NO:26.

15. The composition claim 1, wherein the RNA transcript comprises a sequence selected from the group consisting of SEQ ID NO:12, SEQ ID NO: 13, SEQ ID NO: 14, SEQ ID NO:15, SEQ ID NO:16, SEQ ID NO: 17, SEQ ID NO: 18, SEQ ID NO:19, SEQ ID NO:20, SEQ ID NO:21, SEQ ID NO:22, SEQ ID NO:23, and SEQ ID NO:24.

16.-31. (canceled)

32. A method of treating a subject who has a disease caused by the presence of an SVA mobile element in the subject's genome, the method comprising administering to the subject a therapeutically effective amount of an inhibitory nucleic acid that is complementary to a portion of an RNA transcript derived from the SVA element.

33. The method of claim 32, wherein the RNA transcript comprises a hexanucleotide repeat.

34. The method of claim 33, wherein the hexanucleotide repeat comprises, or is complementary to, the polynucleotide sequence CCCTCT (SEQ ID NO:1).

35. The method of claim 32, wherein the RNA transcript comprises a polynucleotide sequence derived from the SVA element and a polynucleotide sequence derived from the subject's genome.

36. The method of claim 35, wherein the polynucleotide sequence derived from the subject's genome maps to a protein-coding gene.

37. (canceled)

38. The method of claim 32, wherein the disease caused by the presence of an SVA mobile element in the subject's genome is X-linked Dystonia Parkinsonism.

39. (canceled)

40. The method of claim 32, wherein the inhibitory nucleic acid reduces the abundance of the RNA transcript derived from the SVA element.

41. The method of claim 32, wherein the RNA transcript comprises a sequence selected from the group consisting of SEQ ID NO:12, SEQ ID NO: 13, SEQ ID NO:14, SEQ ID NO:15, SEQ ID NO:16, SEQ ID NO: 17, SEQ ID NO: 18, SEQ ID NO:19, SEQ ID NO:20, SEQ ID NO:21, SEQ ID NO:22, SEQ ID NO:23, and SEQ ID NO:24.

42. The method of claim 32, wherein the inhibitory nucleic acid is an antisense oligonucleotide.

43.-59. (canceled)


SEQUENCE LISTING (49 sequences)

SEQ ID NO: 1 - skipped

SEQ ID NO: 2 - DNA, 20 bp, synthetic construct
  source 1..20: mol_type = other DNA
aaatgaaatagctctccctc

SEQ ID NO: 3 - DNA, 20 bp, synthetic construct
  source 1..20: mol_type = other DNA
aaaatgaaatagctctccct

SEQ ID NO: 4 - DNA, 20 bp, synthetic construct
  source 1..20: mol_type = other DNA
aatgaaatagctctccctct

SEQ ID NO: 5 - DNA, 20 bp, synthetic construct
  source 1..20: mol_type = other DNA
atgaaatagctctccctctc

SEQ ID NO: 6 - DNA, 20 bp, synthetic construct
  source 1..20: mol_type = other DNA
aaaaatgaaatagctctccc

SEQ ID NO: 7 - DNA, 20 bp, synthetic construct
  source 1..20: mol_type = other DNA
aaaaaatgaaatagctctcc

SEQ ID NO: 8 - DNA, 20 bp, synthetic construct
  source 1..20: mol_type = other DNA
ttttttccacatctgatgtg

SEQ ID NO: 9 - DNA, 20 bp, synthetic construct
  source 1..20: mol_type = other DNA
gccttattacaatgccagta

SEQ ID NO: 10 - DNA, 20 bp, synthetic construct
  source 1..20: mol_type = other DNA
ctcaagccttattacaatgc

SEQ ID NO: 11 - DNA, 20 bp, synthetic construct
  source 1..20: mol_type = other DNA
ccctcaagccttattacaat

SEQ ID NO: 12 - DNA, 151 bp, Homo sapiens
  source 1..151: mol_type = genomic DNA
gggagagggagagggagagctatttcattttttttttttacacataagatgtggaaaaaaaatgtactggcattgtaataaggcttgagggaggcacatctcacacatgaacgtgaaaacccaatgtcgtcacacttacgtcatcaaaagc

SEQ ID NO: 13 - DNA, 151 bp, Homo sapiens
  source 1..151: mol_type = genomic DNA
gggagagggagagggagagggagagctatttcattttttttttttccacatcagatgtggaaaaaaaatgtactggcattgtaataaggcttgagggaggcacatctcacacatgaacgtgaaaacagatcggaagagcgtcgggtaggga

SEQ ID NO: 14 - DNA, 150 bp, Homo sapiens
  source 1..150: mol_type = genomic DNA
gggagagggagagggagagctatttcattttttttttttccacatcagatgtggaaaaaaaatgtactggcattgtaataaggcttgagggaggcacagctcacacatgaacgtgaaaacccaatgtcgtcacacgtacggcatcctaag

SEQ ID NO: 15 - DNA, 151 bp, Homo sapiens
  source 1..151: mol_type = genomic DNA
gggagagggagagggagagctatttcattttttttttttacacatcagatgtggaaaaaaaatttactggcattgtaataaggcttgagggaggcacatctcacacatgaacgtgaaaacccaaagtagtcacacttacgtcatcctaagc

SEQ ID NO: 16 - DNA, 151 bp, Homo sapiens
  source 1..151: mol_type = genomic DNA
gggagagggagagctatttcattttttttttttccacatcagatgtggaaaaaaaatgtactggcattgtaataaggcttgagggaggcacatctcacacatgaacgtgaaaacccaatgtcgtcacacttacgtcatcataagcttatga

SEQ ID NO: 17 - DNA, 151 bp, Homo sapiens
  source 1..151: mol_type = genomic DNA
ggagagggagagctatttcattttttttttttccacatcatatgtggaaaaaaaatgtactggcattgtaataaggcttgagggaggcacatctcacacatgaacgtgaaaaaccaatgtcgtcacacttacgtaataataagagtatgta

SEQ ID NO: 18 - DNA, 151 bp, Homo sapiens
  source 1..151: mol_type = genomic DNA
gggagagggagagggagagctatttcattttttttttttccacatcagatgtggaaaaaaaatgtactggcattgtaataaggcttgagggaggcacatctcacacatgaacgtgaaaacccaatgtcgtcacacttacgtcatcctaagc

SEQ ID NO: 19 - DNA, 151 bp, Homo sapiens
  source 1..151: mol_type = genomic DNA
gctatttcattttttttttttttccacatcagttgtggaaaaaaaatgtactggcattgtaataaggcttgagggaggcacatctcacacatgaacgtgaaaacccaatgtcgtcacacttacgtcatcataagcttatgaactacaaaaa

SEQ ID NO: 20 - DNA, 150 bp, Homo sapiens
  source 1..150: mol_type = genomic DNA
gaggagagggagagggagagggagagggagagggagagggagagggagagctatttcattttttttttttccacatcagatgtggaaaaaaaatgtactggcattgtaaaaaggcttgagggaggcacatctcacacatgaacgtgaaaa

SEQ ID NO: 21 - DNA, 151 bp, Homo sapiens
  source 1..151: mol_type = genomic DNA
aagagagggagagggagagggagagggagagggagagggagagggagagctatttcattttttttttttccacatcagatgtggaaaaaaaatgtactggcattgtaataaggcttgagggaggcacatctcacacatgaacgtgaaaacc

SEQ ID NO: 22 - DNA, 151 bp, Homo sapiens
  source 1..151: mol_type = genomic DNA
gagagggagagggagagggagagggagagctatttcattttttttttttccacatcagatgtggaaaaaaaatgtactggcattgtaataaggcttgagggaggcacatctcacacatgaacgtgaaaaccccatgtcgtcacacttacgg

SEQ ID NO: 23 - DNA, 151 bp, Homo sapiens
  source 1..151: mol_type = genomic DNA
gatggggagagctatttcattttttttttttccccatcagatgtggaaaaaaaatgtactggcattgtaataaggcttgagggaggcacatctcacacatgaacgtgaaaacccaatgtcgtcacacttacgagatcggaagagcgtcggg

SEQ ID NO: 24 - DNA, 151 bp, Homo sapiens; identical to SEQ ID NO: 23
  source 1..151: mol_type = genomic DNA

SEQ ID NO: 25 - DNA, 20 bp, synthetic construct
  source 1..20: mol_type = other DNA
ccctctccctctccctctcc

SEQ ID NO: 26 - DNA, 12 bp, synthetic construct
  source 1..12: mol_type = other DNA
ccctctccctct

SEQ ID NO: 27 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
aaatgaaatagctctccctc

SEQ ID NO: 28 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified thymine with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified thymine with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified thymine
  misc_feature 1..5: note = RNA
  misc_feature 6..15: note = DNA
  misc_feature 16..20: note = RNA
aaaatgaaatagctctccct

SEQ ID NO: 29 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified thymine with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified thymine
  misc_feature 1..5: note = RNA
  misc_feature 6..15: note = DNA
  misc_feature 16..20: note = RNA
aatgaaatagctctccctct

SEQ ID NO: 30 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified thymine with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified thymine with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
atgaaatagctctccctctc

SEQ ID NO: 31 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified thymine with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
aaaaatgaaatagctctccc

SEQ ID NO: 32 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified thymine with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
aaaaaatgaaatagctctcc

SEQ ID NO: 33 - DNA, 20 bp, synthetic construct
  source 1..20: mol_type = other DNA
  modified_base 1: mod_base = OTHER; note = 5'-terminal 2'-O-methoxyethyl modified thymine with phosphorothioate linkage
  modified_base 2: mod_base = OTHER; note = internal 2'-O-methoxyethyl modified thymine with phosphorothioate linkage
  modified_base 3: mod_base = OTHER; note = internal 2'-O-methoxyethyl modified thymine with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified thymine with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified thymine with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
ttttttccacatctgatgtg

SEQ ID NO: 34 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified thymine with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
gccttattacaatgccagta

SEQ ID NO: 35 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified thymine with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
ctcaagccttattacaatgc

SEQ ID NO: 36 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified thymine
  misc_feature 1..5: note = RNA
  misc_feature 6..15: note = DNA
  misc_feature 16..20: note = RNA
ccctcaagccttattacaat

SEQ ID NO: 37 - DNA, 20 bp, synthetic construct
  source 1..20: mol_type = other DNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified nucleotide with phosphorothioate linkage
  modified_base 6: mod_base = OTHER; note = nucleotide with phosphorothioate linkage
  modified_base 7: mod_base = OTHER; note = nucleotide with phosphorothioate linkage
  modified_base 8: mod_base = OTHER; note = nucleotide with phosphorothioate linkage
  modified_base 9: mod_base = OTHER; note = nucleotide with phosphorothioate linkage
  modified_base 10: mod_base = OTHER; note = nucleotide with phosphorothioate linkage
  modified_base 11: mod_base = OTHER; note = nucleotide with phosphorothioate linkage
  modified_base 12: mod_base = OTHER; note = nucleotide with phosphorothioate linkage
  modified_base 13: mod_base = OTHER; note = nucleotide with phosphorothioate linkage
  modified_base 14: mod_base = OTHER; note = nucleotide with phosphorothioate linkage
  modified_base 15: mod_base = OTHER; note = nucleotide with phosphorothioate linkage
  modified_base 16: mod_base = OTHER; note = internal 2'-O-methoxyethyl modified thymine with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
  misc_feature 1..5: note = RNA
  misc_feature 6..15: note = DNA
  misc_feature 16..20: note = RNA
ccctctccctctccctctcc

SEQ ID NO: 38 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified thymine with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified nucleotide with phosphorothioate linkage
  modified_base 13: mod_base = OTHER; note = internal 2'-O-methoxyethyl modified thymine with phosphorothioate linkage
  modified_base 14: mod_base = OTHER; note = internal 2'-O-methoxyethyl modified nucleotide with phosphorothioate linkage
  modified_base 15: mod_base = OTHER; note = internal 2'-O-methoxyethyl modified thymine with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
aaatgaaatagctctccctc

SEQ ID NO: 39 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified thymine with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified thymine with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified nucleotide with phosphorothioate linkage
  modified_base 13: mod_base = OTHER; note = internal 2'-O-methoxyethyl modified nucleotide with phosphorothioate linkage
  modified_base 14: mod_base = OTHER; note = internal 2'-O-methoxyethyl modified thymine with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified thymine with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified thymine
aaaatgaaatagctctccct

SEQ ID NO: 40 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified thymine with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified thymine with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified thymine with phosphorothioate linkage
  modified_base 13: mod_base = OTHER; note = internal 2'-O-methoxyethyl modified nucleotide with phosphorothioate linkage
  modified_base 14: mod_base = OTHER; note = internal 2'-O-methoxyethyl modified thymine with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified thymine
aatgaaatagctctccctct

SEQ ID NO: 41 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified thymine with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified thymine with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified thymine with phosphorothioate linkage
  modified_base 12: mod_base = OTHER; note = internal 2'-O-methoxyethyl modified nucleotide with phosphorothioate linkage
  modified_base 13: mod_base = OTHER; note = internal 2'-O-methoxyethyl modified thymine with phosphorothioate linkage
  modified_base 14: mod_base = OTHER; note = internal 2'-O-methoxyethyl modified nucleotide with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified thymine with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
atgaaatagctctccctctc

SEQ ID NO: 42 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified thymine with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified thymine with phosphorothioate linkage
  modified_base 12: mod_base = OTHER; note = internal 2'-O-methoxyethyl modified nucleotide with phosphorothioate linkage
  modified_base 13: mod_base = OTHER; note = internal 2'-O-methoxyethyl modified nucleotide with phosphorothioate linkage
  modified_base 14: mod_base = OTHER; note = internal 2'-O-methoxyethyl modified nucleotide with phosphorothioate linkage
  modified_base 15: mod_base = OTHER; note = internal 2'-O-methoxyethyl modified thymine with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified thymine with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
aaaaatgaaatagctctccc

SEQ ID NO: 43 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified thymine with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified thymine with phosphorothioate linkage
  modified_base 13: mod_base = OTHER; note = internal 2'-O-methoxyethyl modified nucleotide with phosphorothioate linkage
  modified_base 14: mod_base = OTHER; note = internal 2'-O-methoxyethyl modified nucleotide with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified thymine with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
aaaaaatgaaatagctctcc

SEQ ID NO: 44 - RNA, 20 nt, synthetic construct
  source 1..20: mol_type = other RNA
  modified_base 1: mod_base = OTHER; note = 5'-terminal 2'-O-methoxyethyl modified thymine with phosphorothioate linkage
  modified_base 2: mod_base = OTHER; note = internal 2'-O-methoxyethyl modified thymine with phosphorothioate linkage
  modified_base 3: mod_base = OTHER; note = internal 2'-O-methoxyethyl modified thymine with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified thymine with phosphorothioate linkage
  modified_base 6: mod_base = OTHER; note = internal 2'-O-methoxyethyl modified thymine with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified thymine with phosphorothioate linkage
  modified_base 13: mod_base = OTHER; note = internal 2'-O-methoxyethyl modified nucleotide with phosphorothioate linkage
  modified_base 14: mod_base = OTHER; note = internal 2'-O-methoxyethyl modified thymine with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified thymine with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
ttttttccacatctgatgtg

SEQ ID NO: 45 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified thymine with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified thymine with phosphorothioate linkage
  modified_base 8: mod_base = OTHER; note = internal 2'-O-methoxyethyl modified thymine with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified nucleotide with phosphorothioate linkage
  modified_base 13: mod_base = OTHER; note = internal 2'-O-methoxyethyl modified thymine with phosphorothioate linkage
  modified_base 14: mod_base = OTHER; note = internal 2'-O-methoxyethyl modified nucleotide with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified thymine with phosphorothioate linkage
  modified_base 20: mod_base = OTHER; note = 3'-terminal 2'-O-methoxyethyl modified nucleotide
gccttattacaatgccagta

SEQ ID NO: 46 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified thymine with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified nucleotide with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified thymine with phosphorothioate linkage
  modified_base 10: mod_base = OTHER; note = internal 2'-O-methoxyethyl modified thymine with phosphorothioate linkage
  modified_base 11: mod_base = OTHER; note = internal 2'-O-methoxyethyl modified nucleotide with phosphorothioate linkage
  modified_base 12: mod_base = OTHER; note = internal 2'-O-methoxyethyl modified thymine with phosphorothioate linkage
  modified_base 13: mod_base = OTHER; note = internal 2'-O-methoxyethyl modified thymine with phosphorothioate linkage
  modified_base 14: mod_base = OTHER; note = internal 2'-O-methoxyethyl modified nucleotide with phosphorothioate linkage
  modified_base 15: mod_base = OTHER; note = internal 2'-O-methoxyethyl modified nucleotide with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified thymine with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified nucleotide
ctcaagccttattacaatgc

SEQ ID NO: 47 - RNA, 20 nt, synthetic construct
  source 1..20: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified nucleotide with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified nucleotide with phosphorothioate linkage
  modified_base 11: mod_base = OTHER; note = internal 2'-O-methoxyethyl modified thymine with phosphorothioate linkage
  modified_base 12: mod_base = OTHER; note = internal 2'-O-methoxyethyl modified thymine with phosphorothioate linkage
  modified_base 13: mod_base = OTHER; note = internal 2'-O-methoxyethyl modified nucleotide with phosphorothioate linkage
  modified_base 14: mod_base = OTHER; note = internal 2'-O-methoxyethyl modified thymine with phosphorothioate linkage
  modified_base 15: mod_base = OTHER; note = internal 2'-O-methoxyethyl modified thymine with phosphorothioate linkage
  modified_base 16: mod_base = OTHER; note = internal 2'-O-methoxyethyl modified nucleotide with phosphorothioate linkage
  modified_base 17: mod_base = OTHER; note = internal 2'-O-methoxyethyl modified nucleotide with phosphorothioate linkage
  modified_base 18: mod_base = OTHER; note = internal 2'-O-methoxyethyl modified nucleotide with phosphorothioate linkage
  modified_base 19: mod_base = OTHER; note = internal 2'-O-methoxyethyl modified nucleotide with phosphorothioate linkage
  modified_base 20: mod_base = OTHER; note = 3'-terminal 2'-O-methoxyethyl modified thymine
ccctcaagccttattacaat

SEQ ID NO: 48 - RNA, 12 nt, synthetic construct
  source 1..12: mol_type = other RNA
  modified_base 1: mod_base = OTHER; note = 5'-terminal 2'-O-methoxyethyl modified nucleotide with phosphorothioate linkage
  modified_base 2: mod_base = OTHER; note = internal 2'-O-methoxyethyl modified nucleotide with phosphorothioate linkage
  modified_base 3: mod_base = OTHER; note = internal 2'-O-methoxyethyl modified nucleotide with phosphorothioate linkage
  modified_base 4: mod_base = OTHER; note = internal 2'-O-methoxyethyl modified thymine with phosphorothioate linkage
  modified_base 5: mod_base = OTHER; note = internal 2'-O-methoxyethyl modified nucleotide with phosphorothioate linkage
  modified_base 6: mod_base = OTHER; note = internal 2'-O-methoxyethyl modified thymine with phosphorothioate linkage
  modified_base 7: mod_base = OTHER; note = internal 2'-O-methoxyethyl modified nucleotide with phosphorothioate linkage
  modified_base 8: mod_base = OTHER; note = internal 2'-O-methoxyethyl modified nucleotide with phosphorothioate linkage
  modified_base 9: mod_base = OTHER; note = internal 2'-O-methoxyethyl modified nucleotide with phosphorothioate linkage
  modified_base 10: mod_base = OTHER; note = internal 2'-O-methoxyethyl modified thymine with phosphorothioate linkage
  modified_base 11: mod_base = OTHER; note = internal 2'-O-methoxyethyl modified nucleotide with phosphorothioate linkage
  modified_base 12: mod_base = OTHER; note = 3'-terminal 2'-O-methoxyethyl modified thymine
ccctctccctct

SEQ ID NO: 49 - DNA, 110 bp, synthetic construct
  source 1..110: mol_type = other DNA
agagggagagctatttcattttttttttttccacatcagatgtggaaaaaaaatgtactggcattgtaataaggcttgagggaggcacatctcacacatgaacgtgaaaa